(19) United States
(12) Patent Application Publication
(10) Pub. No.: US 2008/0103209 A1
(43) Pub. Date: May 1, 2008

(54) Compounds And Methods For Treating Non-Inflammatory Pain Using Ppar Alpha Agonists

(75) Inventors: Daniele Piomelli, Irvine, CA (US); Jesse Loverme, Costa Mesa, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of The University of California, Oakland, CA (US)

(21) Appl. No.: 11/587,100

(22) PCT Filed: Apr. 22, 2005
(86) PCT No.: PCT/US05/13858

§ 371 Date: Nov. 12, 2007

Related U.S. Application Data
(60) Provisional application No. 60/565,196, filed on Apr. 23, 2004.

(51) Int. Cl. A61K 31/16; A61P 25/00

(52) U.S. Cl. 514/627; 514/625

(57) ABSTRACT

Compositions and methods for treating noninflammatory pain, including but not limited to, neuropathic pain by using peroxisome proliferator activated receptor a (PPARa) agonists to treat a subject having such pain are described. The agonists may be used with additional therapeutic agents such as an inhibitor of fatty acid amide hydrolase or a cannabinoid CB1 or CB2 cannabinoid receptor agonist.

### CROSS-REFERENCES TO RELATED APPLICATIONS

This application contains subject matter related to that of U.S. patent application Ser. No. 10/112,509 filed Mar. 27, 2002; U.S. Provisional Application No. 60/336,289 filed Oct. 31, 2001; U.S. Patent Application No. 60/279,542 filed Mar. 27, 2001, U.S. Provisional Application No. 60/485,062 filed Jul. 2, 2003, and U.S. patent application Ser. No. 10/681,858 filed Oct. 7, 2003. The contents of each of the above-referenced applications is incorporated herein by reference in its entirety.

### BACKGROUND OF THE INVENTION

Pain may be classified into three categories according to its neurophysiology and presentation. Nociceptive pain provides a well familiar initial alert to the presence of a noxious stimulus capable of causing injury. Nociceptive pain prompts a protective withdrawal from the stimulus. The nociceptive pain signal involves the direct transmission of the noxious stimulus from the nociceptor primary afferent (A-delta and C-fibers) through the dorsal horn of the spinal cord, via ascending sensory tracts to the thalamus and cortex, where the stimulus is perceived as painful. Often, no tissue damage occurs as withdrawal from the stimulus prevents tissue injury.

Inflammatory pain develops in response to tissue damage occurring from the noxious stimuli. In response to the tissue injury, cytokines and other mediators are released which strengthen nociception. As a result primary hyperalgesia (increased sensitivity to pain) occurring in the area of injury and a secondary hyperalgesia occurring in the tissue surrounding the injury ensue. The hyperalgesia subsides with the inflammation as the tissue is healed.

Inflammatory pain is typically treated with non-steroidal anti-inflammatory drugs (NSAIDs) such as aspirin. It has recently been proposed that cannabinoid mimetics may also be useful in the treatment of inflammatory pain. Due primarily to their anti-inflammatory properties, PPARα agonists have also been reported to be useful, alone or in combination with other anti-inflammatory agents, in treating inflammation and inflammatory pain.

Neuropathic pain differs from acute pain not only in its onset and duration but also in its underlying mechanisms. In neuropathic pain, the sensation of pain is chronic and persists for months to years and may no longer relate to the presence of any significant inflammation of the innervated tissue. Neuropathic pain is pain that is principally mediated by peripheral or central nerve damage or dysfunction that results in chronic hyperalgesia, allodynia (a painful sensation of a normally non-painful stimulus), and/or spontaneous pain. Neuropathic pain is initiated, caused, or maintained by a primary lesion or dysfunction of the nervous system. The primary lesion may be due to traumatic injury to a nerve. The dysfunction may result from a prolonged and intense bout of inflammatory pain, which alters the sensitivity of the nervous system to nociception even after the healing of the initial lesion is completed. Such neuropathic pain often involves a peripheral and central sensitization to pain due to altered afferent input. Thus, neuropathic pain is often not linked to any concurrent proportionate or identifiable noxious stimulus or any proportionate or readily identifiable concurrent inflammation amenable to therapy with an anti-inflammatory agent.

Peroxisome proliferator activated receptors (PPAR) are a family of transcription factors and have been principally studied with respect to lipid homeostasis. Three PPAR subtypes have been identified: α, β (also described as δ), and γ. All three subtypes have domain structure common with other members of the nuclear receptor family. DNA-binding domains are highly conserved among PPAR subtypes, but ligand binding domains are less well conserved. (Willson et al., J. Med. Chem. 43:527 (2000)). PPARs bind to RXR transcription factors to form heterodimers that bind to DNA sequences containing AGGTCAnAGGTCA. It has been shown that ligand binding to PPAR can induce gene expression.

PPARγ is the best characterized of the three subtypes. Activation of PPARγ promotes adipocyte differentiation by repressing expression of the ob and TNFα genes. Activation of PPARγ also results in in vivo insulin sensitization. PPARγ has been implicated in several diseases including diabetes, hypertension, dyslipidemia, inflammation, and cancer.

PPARα is well known for its metabolic and anti-inflammatory roles. PPARα is expressed at high levels in the liver, heart, renal cortex, brown fat, and intestine. PPARα regulates genes involved in almost all aspects of lipid metabolism and has been postulated to play a role in dyslipidemia, atherosclerosis, obesity, inflammation, and diabetes. Recently PPARα has been reported to inhibit inflammatory edema and inflammatory pain (see Taylor et al. Inflammation 26(3):121 (2002) and Sheu et al. J. Invest. Dermatol. 118:94 (2002)).

PPARβ(δ) is the most widely expressed subtype and the least understood. PPARβ(δ) regulates acyl-coA synthetase 2 expression and is postulated to play a role in dyslipidemia, fertility, bone formation, and colorectal cancer. PPARβ(δ) expression in cells reduces their proliferation rate, but PPARβ expression in cells in conjunction with exposure to fatty acids increases proliferation rate.

All three subtypes are postulated to play a role in lipid homeostasis, but comparative studies have demonstrated significant differences among the subtypes. For example, mRNA expression of PPARα and PPARAγ is increased in ob/ob and db/db mice, but mRNA expression of PPARβ(δ) in ob/ob and db/db mice is the same as in control mice. It has also been shown that some ligands that bind to PPARγ and PPARα do not bind or activate PPARβ(δ).

As stated above, the PPAR family has been described as playing a role in obesity. Natural and synthetic subtype specific ligands have been identified for PPARα, PPARAγ, and PPARβ(δ). PPARα-selective compounds typically have an enhanced ability to reduce body fat and modulate fatty acid oxidation compared to PPARβ or PPARγ selective compounds. PPARα is activated by a number of medium and long-chain fatty acids. PPARα is also activated by compounds known as fibric acid derivatives. These fibric acid derivatives, such as clofibrate, fenofibrate, bezafibrate, ciprofibrate, beclofibrate and etofibrate, as well as gemfibrozil reduce plasma triglycerides along with LDL cholesterol, and they are primarily used for the treatment of hypertriglyceridemia.

Fatty acid ethanolamides (FAE) are unusual components of animal and plant lipids, and their concentrations in non-stimulated cells are generally low (Bachur et al., J. Biol. Chem., 240:1019-1024 (1965); Schmid et al., Chem. Phys. Lipids, 80:133-142 (1996); Chapman, K. D., Chem. Phys. Lipids, 108:221-229 (2000)). FAE biosynthesis can be rapidly enhanced, however, in response to a wide variety of physiological and pathological stimuli, including exposure to fungal pathogens in tobacco cells (Chapman et al., Plant Physiol., 116:1163-1168 (1998)), activation of neurotransmitter receptors in rat brain neurons (Di Marzo et al., Nature, 372:686-691 (1994); Giuffrida et al., Nat. Neurosci., 2:358-363 (1999)) and exposure to metabolic stressors in mouse epidermal cells (Berdyshev et al., Biochem. J, 346:369-374 (2000)). The mechanism underlying stimulus-dependent FAE generation in mammalian tissues is thought to involve two concerted biochemical reactions: cleavage of the membrane phospholipid, N-acyl phosphatidylethanolamine (NAPE), catalyzed by an unknown phospholipase D; and NAPE synthesis, catalyzed by a calcium ion- and cyclic AMP-regulated N-acyltransferase (NAT) activity (Di Marzo et al., Nature, 372:686-691 (1994); Cadas et al., J. NeuroSci., 6:3934-3942 (1996); Cadas et al., H., J. Neurosci., 17:1226-1242 (1997)).

The fact that both plant and animal cells release FAEs in a stimulus-dependent manner suggests that these compounds may play important roles in cell-to-cell communication. Further support for this idea comes from the discovery that the polyunsaturated FAE, anandamide (arachidonylethanolamide), is an endogenous ligand for cannabinoid receptors (Devane et al., Science, 258:1946-1949 (1992))—G protein-coupled receptors expressed in neurons and immune cells, which recognize the marijuana constituent Δ⁹-tetrahydrocannabinol (Δ⁹-THC) (for review, see reference (Pertwee, R. G., Exp. Opin. Invest. Drugs, 9:1553-1571 (2000)).

Two observations make it unlikely that other FAEs also participate in cannabinoid neurotransmission. The FAE family is comprised for the most part of saturated and monounsaturated species, such as palmitoylethanolamide and oleoylethanolamide, which do not significantly interact with cannabinoid receptors (Devane et al., Science, 258:1946-1949 (1992); Griffin et al., J. Pharmacol. Exp. Ther., 292:886-894. (2000)). Second, when the pharmacological properties of the FAEs have been investigated in some detail, as is the case with palmitoylethanolamide, such properties have been found to differ from those of Δ⁹-THC and to be independent of activation of known cannabinoid receptor subtypes (Calignano et al., Nature, 394:277-281 (1998)). Thus, the biological significance of the FAEs remains an intense area of study.

Fatty acid amide hydrolase (FAAH) is the enzyme primarily responsible for the hydrolysis of anandamide in vivo. It also is responsible for the hydrolysis of OEA in vivo. Inhibitors of the enzyme are well known to one of ordinary skill in the art (Cravatt, B. F. et al., Nature, 384:83-87 (1996); Patricelli, M. P. et al., Biochemistry, 38:9804-9812 (1999); WO Patent Publication No. 98/20119; Rodríguez de Fonseca, et al. Nature, 414:209-212 (2001); Calignano, et al., Nature, 394:277-281 (1998)). Mutant mice lacking the gene encoding for FAAH cannot metabolize anandamide (Cravatt, B. F. et al., Proc. Natl. Acad. Sci. U.S.A., 98:9371-9376 (2001)) and, though fertile and generally normal, show signs of enhanced anandamide activity at cannabinoid receptors, such as reduced pain sensation (Cravatt, B. F. et al., Proc. Natl. Acad. Sci. U.S.A., 98:9371-9376 (2001)).

Oleoylethanolamide (OEA) is a natural analogue of the endogenous cannabinoid anandamide. Like anandamide, OEA is produced in cells in a stimulus-dependent manner and is rapidly eliminated by enzymatic hydrolysis, suggesting a role in cellular signaling. However, unlike anandamide, OEA does not activate cannabinoid receptors.

Palmitoylethanolamide (PEA) produces profound analgesia [Calignano, et al., Nature 394, 277-81 (1998); Calignano, et al, Eur J Pharmacol 419, 191-8. (2001); Jaggar, et al., Pain 76, 189-99 (1998)] and anti-inflammation [Benvenuti, et al., Boll Soc It Biol Sper 44, 809-813 (1968); Facci, et al., Proc Natl Acad Sci USA 92, 3376-80 (1995); Mazzari, et al., Eur J Pharmacol 300, 227-36 (1996); Costa, et al., Br J Pharmacol 137, 413-20. (2002); Conti, et al., Br J Pharmacol 135, 181-7. (2002); Lambert, et al., Curr Med. Chem. 9, 663-74 (2002)]. However, the mechanism by which these effects occur has remained undefined. PEA shares structural homology with the endogenous cannabinoid agonist anandamide (arachidonylethanolamide), which too displays peripheral antinociceptive and anti-inflammatory properties, therefore, it has been proposed that the analgesic and anti-inflammatory actions of PEA may occur through activation of peripheral cannabinoid subtype 2 (CB₂) receptors. This evidence is supported by two reports. First, it was initially reported that PEA displaced the binding of the cannabinoid agonist [³H]WIN 55, 212-2 to basophilic leukemia cell membranes. This evidence is highly debatable, as many investigators have subsequently failed to demonstrate binding of PEA to CB₂ receptors. Second, it has been reported that the CB₂ antagonist/inverse agonist SR144528, but not the CB₁ antagonist SR141716A (rimonabant), inhibits the antinociceptive and anti-inflammatory actions of PEA.

PEA has been reported as being an endocannabinoid having a cannabinoid CB₂ agonist-like effect on acute and neuropathic pain. The CB₂ cannabinoid receptor antagonist has been reported to block the analgesic effects of PEA in both an inflammatory pain model and neuropathic pain model (see, Helyes et al. Life Sciences 73:2345 (2003)). Upon investigation of the effects of PEA on pain, as reported herein, we have surprisingly discovered that, notwithstanding the reported ability of the CB2 receptor antagonist SR144528 to block the effects of PEA on such pain, the effects of PEA on pain are mediated by PPARα receptor activation and, indeed, that other PPARα agonists, are found to be effective in treating pain.

Over time, neuropathic pain exacts a substantial toll on the physical, emotional, social and economic well being of an affected individual. Unfortunately, neuropathic pain is more refractory to medication than acute pain. Thus, while a number of agents (lidocaine, capsaicin, opioids, cannabinoids) have been reported to have some limited efficacy in treating neuropathic pain, the pharmacopoeia for the treatment of such pain is relatively bare.

Thus, there is a need for effective medicinal therapies for treating neuropathic pain. Our invention relates to our discovery that PPARα activation is a particularly effective means of treating acute and persistent pain of non-inflammatory origin, including neuropathic pain. To meet the need for new therapies to treat such pain, this invention provides methods and pharmaceutical compositions for treating neuropathic pain by local, topical, systemic and other means administration of a PPARα agonist.

### BRIEF SUMMARY OF THE INVENTION

In a first aspect, the invention provides methods of treating a mammalian subject for noninflammatory pain by administering to the subject a PPARα agonist other than PEA in a therapeutically effective amount. In some embodiments the noninflammatory pain is neuropathic pain, or pain initiated or caused by a primary lesion or dysfunction of the nervous system. The invention provides methods of treating all forms of neuropathic pain, including but not limited to, spontaneous pain, allodynia, and hyperalgesia.

In some embodiments, the noninflammatory pain is a neuropathic pain selected from the group consisting of post trigeminal neuralgia, neuropathic low back pain, peripheral or polyneuropathic pain, complex regional pain syndrome, causalgia, and reflex sympathetic dystrophy, diabetic neuropathy, toxic neuropathy, and chronic neuropathy caused by chemotherapeutic agents.

In some embodiments, the noninflammatory pain is renal and liver colic pain or fibromyalgia.

In some neuropathic pain embodiments, the primary lesion or dysfunction of the nervous system is caused by a mechanical injury to a nerve of the subject. In further embodiment, the mechanical injury is due to compression of a nerve, transection of nerve, causalgia, spinal cord injury, post surgical pain, phantom limb pain, or scar formation in the subject.

In yet other embodiments, the primary lesion or dysfunction of the nervous system is a toxic injury to the nervous system. In some further embodiments, the toxic injury is caused by chemotherapy or exposure to an environmental or occupational toxins. Examples of chemotherapeutic agents causing such injuries include, but are not limited to, vincristine, ciplatin, and taxol. Examples of environmental or occupational toxins causing such injuries include, but are not limited to, lead, thallium, and arsenic. In still other embodiments, the primary lesion or dysfunction of the nervous system is a radiation injury to the nervous system.

Metabolic and nutritional disorders can cause a primary lesion or dysfunction of the nervous system responsible for neuropathic pain. In some embodiments, the primary lesion or dysfunction of the nervous system is a diabetic neuropathy, pellagric neuropathy, alcoholic neuropathy, Beriberi neuropathy, or burning feet syndrome.

In still other embodiments, the subject has a neurological or other disease causing the neurogenic pain. These neurological diseases, include but are not limited to, multiple sclerosis, trigeminal neuralgia, Guillain-Barre syndrome, Fabry's disease, and Tangier disease which are thought to cause a primary lesion or dysfunction of the nervous system. In other embodiments, the other disease is cancer and the pain is cancer pain.

In particular embodiments, the neuropathic pain is a complex regional pain syndrome, sciatica, or diabetic neuropathy.

In some embodiments, the PPARα agonist is a selective for the PPARα receptor over the PPARβ, PPARγ, or PPARδ receptor. In other embodiments, the PPARα agonist is not an agonist or activator of a cannabinoid CB₁ or CB₂ receptor. In still other embodiments, the PPARα agonist is a fatty acid alkanolamide. In still further embodiments, the PPARα agonist is a fatty acid alkanolamide, homologue or analogue other than PEA. In another embodiment the PPARα agonist is OEA. In another set of embodiments, the PPARα agonist is clofibrate or a derivative of clofibrate. Such derivatives would include, but not be limited to, clofibrate; fenofibrate, bezafibrate, gemfibrozil, and ciprofibrate.

In some embodiments, the PPARα agonist has a specificity for PPARα over each or any one of PPARγ and PPARβ that is at least five-fold, ten-fold, twenty-fold or one-hundred fold. In some embodiments, the PPARα agonist has a specificity for PPARα over each or any one of the cannabinoid CB1 or CB2 receptors that is at least five-fold, ten-fold, twenty-fold or one-hundred fold. In some embodiments, the PPARα selective agonist has a half maximal effect at a concentration less than 1 micromolar, 100 nanomolar or 1 nanomolar, or between 1 micromolar and 10 nanomolar. In some embodiments, the compound is an OEA-like compound, including but not limited to, a fatty acid alkanolamide other than PEA. In some embodiments, the subject is a mammal. In some further embodiments, the subject is a human, mouse, rat, rabbit, hamster, guinea pig or primate.

In particular embodiments, the PPARα modulator or OEA-like compound is a compound having the formula:

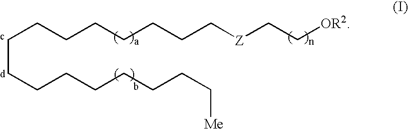

or its pharmaceutically acceptable salt with the proviso that the compound is not PEA.

In this formula, n is from 0 to 5 and the sum of a and b can be from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of substituted or unsubstituted alkyl, hydrogen, substituted or unsubstituted C₁-C₆ alkyl, substituted or unsubstituted lower (C₁-C₆) acyl, homoalkyl, and aryl. Up to eight hydrogen atoms of the compound may also be substituted by methyl or a double bond. In addition, the molecular bond between carbons c and d may be unsaturated or saturated.

In other embodiments, the fatty acid moiety of the fatty acid alkanolamide or ethanolamide compound, homologue, or analog may be saturated or unsaturated, and if unsaturated may be monounsaturated or polyunsaturated.

In some embodiments, the fatty acid moiety of the fatty acid alkanolamide compound, homologue, or analog is a fatty acid selected from the group consisting of oleic acid, palmitic acid, elaidic acid, palmitoleic acid, and linoleic acid. In certain embodiments, the fatty acid moieties have from twelve to 20 carbon atoms. In other further embodiments of the above, the alkanolamide is ethanolamide or propanolamide.

Other embodiments are provided by varying the hydroxyalkylamide moiety of the fatty acid amide compound, homologue or analog. These embodiments include the introduction of a substituted or unsubstituted lower (C₁-C₃) alkyl group on the hydroxyl group of an alkanolamide or ethanolamide moiety so as to form the corresponding lower alkyl ether. In another embodiment, the hydroxy group of the alkanolamide or ethanolamide moiety is bound to a carboxylate group of a C₂ to C₆ substituted or unsubstituted alkyl carboxylic acid to form the corresponding ester of the fatty acid ethanolamide. Such embodiments include fatty acid alkanolamide and fatty acid ethanolamides in ester linkage to organic carboxylic acids such as acetic acid, propionic acid, and butanoic acid. In one embodiment, the fatty acid alkanolamide is oleoylalkanolamide. In a further embodiment, the fatty acid alkanolamide is oleoylethanolamide.

In still another embodiment, the fatty acid ethanolamide compound, homologue, or analog further comprises a substituted or unsubstituted lower alkyl (C₁-C₃) group covalently bound to the nitrogen atom of the fatty acid ethanolamide.

In still other aspects, a preferred compound of the invention is a fatty acid alkanolamide, or homologs and analogs, thereof, which is a selective agonist of the PPARα receptor. Preferred compounds include, but are not limited to, a fatty acid alkanolamide or compound of formula I which provides a half-maximal modulatory effect on the PPARα receptor at a concentration which is at least 5-fold, 10-fold, 50-fold, or 100-fold lower than the concentration of the compound which provides a half-maximal effect (or no effect) on a PPARβ or PPARγ receptor from the same species of origin as the PPARα receptor under comparable assay conditions (e.g., same in vivo test species and conditions or same pH, same buffer components). Still further preferred PPARα agonist compounds, including OEA-like compounds, have a half maximal modulatory effect on the receptor at a concentration of less than 1 micromolar, less than 100 nanomolar, and more preferably less than 10 nanomolar.

Still other aspects of the invention address methods of using and administering selective high affinity (high affinity indicates an ability to produce a half-maximal effect at a concentration of 1 micromolar or less) agonists of PPARα for treating neuropathic pain in mammals (e.g., humans, primates, cats, dogs). The subject compositions may be administered by a variety of routes, including orally. In some embodiments, the selective high affinity agonists of PPARα are OEA-like compounds, including, but not limited to, fatty acid alkanolamides and the compounds according to Formula I above and Formula VI below.

In still other aspects of the invention, a Fatty Acid Amide Hydrolase (FAAH) inhibitor is administered (e.g., alone or in combination with any one or more of a PPARα agonist, a CB1 agonist, or a anandamide transport inhibitor) to treat a condition or disease in a subject mediated by PPARα or responsive to therapy with a PPARα agonist (e.g, neuropathic pain, noninflammatory pain). In some embodiments, the PPARα agonist is an OEA-like compound, including, but not limited to a compound of Formula I or Formula VI. In some further embodiments, the FAAH inhibitor is administered to a subject also receiving a PPARα agonist, including but not limited to an agonist of Formula I, Formula VI, and particularly, selective PPARα agonists. In preferred embodiments, the subject is human. In some embodiments, the OEA-like modulator is an agonist of PPARα and the disease or condition to be treated is neuropathic pain. The FAAH inhibitor may work by inhibiting the FAAH-mediated hydrolysis of an administered OEA like compound subject to such hydrolysis and/or by inhibiting the hydrolysis of endogenously formed OEA or another endogenous FAAH substrate which is a PPARα agonist. In a preferred embodiment, the FAAH inhibitor is administered with a OEA-like compound subject to hydrolysis by FAAH so that the biological half-life of the OEA like compound is increased. In some embodiments, the OEA-like compound is OEA. In some embodiments, the FAAH inhibitor is co-administered with an anandamide transport inhibitor selected from the group consisting of M404, AM1172, OMDM1, and UCM707.

In yet a third aspect, the invention provides a method of treating neuropathic pain by administering both a CB₁ cannabinoid agonist and an agent which increases PPARα activation in vivo (e.g., a PPARα agonist other than PEA, a FAAH inhibitor, inhibitor) to a subject having such pain. In some embodiments, the PPARα agonist is OEA. In some embodiments, the CB1 cannabinoid agonist is anandamide. In yet other embodiments, the CB₁ cannabinoid agonist is anandamide and the PPARα agonist is OEA. In another embodiment, the PPARα agonist is clofibrate or a derivative of clofibrate. Such derivatives would include, but are not limited to, clofibrate; fenofibrate, bezafibrate, gemfibrozil, and ciprofibrate. In a further embodiment, the compound is a PPARα agonist and optionally is also a modulator (e.g. an activator or agonist) of a cannabinoid receptor (e.g., CB1 or CB2 receptors).

In yet a fourth aspect, the invention provides a method of treating noninflammatory or neuropathic pain by administering both an anandamide transport inhibitor and an agent which increases PPARα activation in vivo (e.g., a PPARα agonist other than PEA, a FAAH inhibitor) to a subject having such pain. In some embodiments, the PPARα agonist is OEA. In some embodiments, the anandamide transport inhibitor is M404, AM1172, OMDM1, or UCM707. In yet other embodiments, the anandamide transport inhibitor is one of M404, AM1172, OMDM1 or UCM707 and the PPARα agonist is OEA.

The pharmaceutically active agents (e.g., FAAH inhibitors, PPARα agonists, and CB1 cannabinoid agonists, anandamide transport inhibitors) to be used according to the invention may be administered by a variety of routes. These routes include, but are not limited to, the oral route, the intravenous route, and the dermal routes of administration. They may be administered locally (e.g., near the site of the pain or the primary lesion or dysfunction) or systemically. When one or more active agents are to be administered, they may be administered concurrently or at different times. They may be administered on the same or different schedules (e.g., according to the biological half-times in the body or their individual duration of action). They may be administered together via one pharmaceutical composition or via separate pharmaceutical compositions.

In still another aspect, the invention provides methods for treating nociceptive, non-inflammatory pain by administration of a PPARα agonist, FAAH inhibitor, and CB1 cannabinoid agonist, either individually or in any combination thereof, in the treatment of such pain to a mammal having such pain.

In one additional aspect the invention provides a method of treating a subject for neuropathic pain by determining if the subject has chronic pain; if the chronic pain is resistant to therapy with a non-steroidal, anti-inflammatory agent other than a PPARα agonist, and administering a PPARα agonist to the subject. In another aspect, the invention uses a PPARα agonist in the manufacture of a medicament for treating a subject having a chronic pain or neuropathic pain. In a further embodiment, such pain is a pain resistant to treatment by a non-steroidal anti-inflammatory agent other than a PPARα agonist.

In all aspects of the invention, and embodiments thereof, setting forth a class of PPARα agonist(s) to be used according to the invention, in a further one of each such embodiments, the PPARα agonist is not PEA. In another further one of such embodiments, the PPARα agonist is subject to the proviso that PPARα agonist is not a CB2 cannabinoid agonist or a CB1 cannabinoid agonist. In another further one of such embodiments, the PPARα agonist is subject to the proviso that PPARα agonist is not a fatty acid amide.

In all such above aspects of the invention, and embodiments thereof, setting forth a CB1 cannabinoid receptor agonist, in a further embodiment thereof, the CB1 cannabinoid receptor agonist is CP-55940, Win-55212-2, anandamide, methanandamide, or 2-arachidonoylglycerol.

In all such above aspects of the invention, and embodiments thereof, setting forth a FAAH inhibitor, in one further embodiment the FAAH inhibitor is Compound M, URB597 and AM374 or a haloenol lactone as taught in U.S. Pat. No. 6,525,090.

In all such above aspects of the invention, and embodiments thereof, setting forth use of a PPARα agonist, in one further such embodiment the PPARα agonist (e.g., alone or in combination with a CB1 agonist and/or FAAH inhibitor) is administered to prevent neuropathic pain and the schedule and dosage of medication(s) (e.g, the active agents for use according to the invention) is determined to be sufficient and/or adjusted to control such pain. Such adjusting may be made upon subjective or objective evaluations of the pain. In some such embodiments, the PPARα agonist (e.g., alone or in combination with a CB1 agonist and/or FAAH inhibitor and/or an anandamide transport inhibitor) is specifically administered to prevent neuropathic pain.

In all aspects of the invention, and embodiments thereof, setting forth a subject to which the PPARα agonist(s) is to be administered, in a further one of each such embodiments, the subject is a human with the proviso that the subject is not otherwise in need for treatment with a PPARα agonist. In some such embodiments, the subject does not have a condition treatable with a PPARα agonist. In some such embodiments, the absent condition is selected from the group of obesity, overweight, an appetite disorder, an eating disorder, an appetite disorder, excess body fat, a metabolic disorder, cellulite, Type II diabetes, insulin resistance, Type I diabetes, Syndrome X, hyperlipidemia, hypercholesterolemia, hypertriglyceridemia, artherogenesis. In other such embodiments, the absent condition is an inflammatory disorder or condition, Alzheimers disease, Crohn's disease, a vascular inflammation, an inflammatory bowel disorder, an immune disorder, autoimmunity, environmental immunity, rheumatoid arthritis, asthma, or thrombosis.

In all aspects of the invention, and embodiments thereof, setting forth a subject to which the PPARα agonist(s) is to be administered, in a further one of each such embodiments, the subject is a human with the proviso that the subject does not have a gastrointestinal disease or a gastrointestinal disease needing treatment with a PPARα agonist. In some further such embodiments, the disease is an inflammatory bowel disease, gastric ulcer, gastric varices, Crohn's disease, gastritis, irritable bowel syndrome and ulcerative, colitis, or GI cancer.

In all aspects of the invention, and embodiments thereof, setting forth a subject to which the PPARα agonist(s) is to be administered, in a further one of each such embodiments, the subject is a human with the proviso that the subject does not have an inflammatory disease needing treatment with a PPARα agonist. In some further embodiments, the patient does not have an infectious disease needing PPARα agonist treatment. In some further embodiments, the infectious disease is herpes simplex infection, herpes zoster infections, or HIV.

In all aspects of the invention, and embodiments thereof, setting forth a subject to which the PPARα agonist(s) is to be administered, in a further one of each such embodiments, the subject is a human with the proviso that the subject does not have an inflammation requiring PPARα agonist therapy. In some further embodiments, the inflammation is associated with pulmonary edema, kidney stones, minor injuries, wound healing, skin wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, type II diabetes, myasthenia gravis, multiple sclerosis, sarcoidosis, nephrotic syndrome, Behcet's syndrome, polymyositis, gingivitis, hypersensitivity, swelling occurring after injury, or myocardial ischemia.

In all aspects of the invention, and embodiments thereof, setting forth a subject to which the PPARα agonist(s) is to be administered, in a further one of each such embodiments, the subject is a human with the proviso that the subject does not have eye or ophthalmic disease needing treatment with a PPARα agonist. In some further embodiments, does not have retinitis, retinopathies, conjunctivitis, uveitis, ocular photophobia, and of acute injury to the eye tissue.

In all aspects of the invention, and embodiments thereof, setting forth a subject to which the PPARα agonist(s) is to be administered, in a further one of each such embodiments, the subject is a human with the proviso that the subject does not have a pulmonary inflammation needing treatment with a PPARα agonist. In some further embodiments, the absent pulmonary inflammation is associated with viral infections and cystic fibrosis.

In some embodiments, the subject is a human patient with the proviso that the subject does not have a cortical dementia (e.g., Alzheimer's disease).

In another set of embodiments, wherein the subject is a human treated for a non-inflammatory pain (e.g., neuropathic pain), the dose of the PPARα agonist is adjusted to control (reduce the frequency or severity or both the frequency and severity) of the noninflammatory pain. In further such embodiments, the subject does not have a condition giving rise to any, essentially any, or an appreciable amount of inflammatory pain.

In all aspects of the invention, for embodiments thereof setting forth the use or presence of one or more active agents selected from the group consisting of PPARα agonists, FAAH inhibitors, CB1 or CB2 cannabinoid agonists, and anandamide transport inhibitors, in one further embodiment of each of such, there is a proviso that each of the active agents set forth is not PEA. In another embodiment of each of such, there is a proviso that each of the active agents set forth is not OEA. In another embodiment of each of such, there is a proviso that each of the active agents set forth is not anandamide. In another embodiment of each of such, there is a proviso that each of the active agents set forth is not a fatty acid amide. In another embodiment of each of such, there is a proviso that each of the active agents set forth, or at least one of such, is not an activator, agonist, antagonist or inhibitor of the vanilloid receptor in vivo and/or in vitro.

In some embodiments, the noninflammatory or neuropathic pain is one which is resistant to treatment with a non-steroidal anti-inflammatory agent. In some embodiments, the NSAID is acetaminophen or COX-2 inhibitor.

In another aspect the invention is drawn to synergism between the endogenous cannabinoid systems effecting pain relief and pain relief provided by activation of PPARα. In this aspect, the PPARα agonist is co-administered separately or together with a cannabinoid CB1 or CB2 receptor agonist to effect pain relief for neuralgia or any of the conditions recited above. In one embodiment, a dual PPARα agonist cannabinoid receptor agonist (e.g., CB1 and/or CB2) can be administered.

In all aspects of the invention, and embodiments thereof, setting forth a subject to which the PPARα agonist(s) is to be administered, in a further one of each such embodiments, the PPARα agonist acts to relieve pain essentially or primarily via activation or agonism of the PPARα receptor.

### BRIEF DESCRIPTION OF THE DRAWINGS

FIG. 1. High-affinity PPARα agonists inhibit acute nociception. Effects of intraplantar (i.pl.) administration of PEA (circles), GW7647 (squares), Wy-14643 (triangles) or fenofibric acid (diamonds) on (A) first-phase (phase I) or (B) second-phase (phase II) of formalin-evoked pain behavior in Swiss mice. Baseline formalin responses were: PEA, phase I: 161±19 s; phase II: 148±21 s; GW7647, I: 176±12 s; II: 197±18 s; Wy-14643, I: 137±8 s; II: 154±13; fenofibric acid, I: 165±19 s; II: 163±31 s (n=6-10). Effects of i.pl. administration of vehicle (open bars), (C) GW501516 or (D) ciglitazone (closed bars, each at 50 μg) on phase I and II formalin pain in wild-type C57BL6 mice (n=6-10). Effects of vehicle (open bars), (E) GW7647 (closed bars; 50 μg, i.pl.), (F) PEA (closed bars; 50 μg, i.pl.), or (G) methanandamide (m-AEA; closed bars; 50 μg, i.pl.) on formalin pain in wild-type C57BL6 (+/+) or PPARα^{-/-} (−/−) mice (n=6-19). (H) Effects of subcutaneous (s.c.) administration of PEA (circles), GW7647 (squares) or Wy-14643 (triangles) on magnesium sulfate-evoked writhing in Swiss mice (n=8). (I) Effects of s.c. administration of vehicle (V) or PEA (20 mg-kg⁻) on magnesium sulfate writhing in wild-type C57BL6 (+/+) and PPARα^{-/-} (−/−) mice (n=10). (*, P<0.05; ** P<0.01; P<0.001) vs. V; means ±SEM.

FIG. 2. The anti-inflammatory effects of PEA are abolished in PPARα^{-/-} mice. a and b, Effects vehicle (open circles, saline/PEG/Tween80 (901515)), PEA (closed circles; 10 mg kg⁻¹) or WY-14643 (closed squares; 20 mg kg⁻¹) 30 minutes prior to carrageenan-evoked (2% w v⁻¹, 20 μl/paw, intraplantar) paw edema in wild type (a) or PPAR^{-/-}(b) mice *, P<0.05, **, P<0.01, ANOVA followed by Dunnett's test; ^{##}P<0.001 vs. wildtype GW7647, student's t-test (n=5-6). c, Dose-dependent anti-inflammatory effects of topically applied PEA (closed circles) or palmitic acid (closed squares, 10 μg) on 12-O-tetradecanoylphorbol-13-acetate (TPA)-induced ear edema in C57BL/6J mice (n=5-7). d and e, Effects of vehicle (V), PEA (PEA; 12 μg) or GW7647 (GW; 20 μg) in the mouse ear edema assay in wild type (b) or PPAR^{-/-}(c) mice *, P<0.05, **, P<0.01, ANOVA followed by Dunnett's test (n=6-16).

FIG. 3. PEA selectively activates PPARα. a, Concentration-dependent activation of human PPARα by PEA (closed circles) and GW7647 (closed squares), but not palmitic acid (open circles). b, PEA activates human PPARα (closed circles), but not PPARβ (open circles) and PPARγ (closed squares) (n=12). c, PEA (450 μg) or GW7647 (755 μg) topically applied to inflamed mouse skin induces the expression of PPARα mRNA. Data is expressed as arbitrary units, normalized to glyceraldehyde 3-phosphate dehydrogenase (GAPDH) mRNA levels. *, P<0.05, **, P<0.01, ANOVA followed by Dunnett's test (n=4-5). d, PEA levels in untreated mouse skin (UN, shaded bars), or following administration of either vehicle (V, acetone, open bars) or TPA (black bars) at either 4 or 18 hours following treatment. *, P<0.05, **, P<0.01, ANOVA followed by Dunnett's test (n=4-5).

FIG. 4. PPARα selectively modulates pain through heat shock protein 90 (hsp90). a and b, Inhibition of PEA (PEA; 50 μg/paw) antinociception by geldanamycin (GEL; 10 μg/paw) on the early (a) and late (b) phases of formalin-evoked (vehicle, V) nociception in Swiss mice. **, P<0.01, ANOVA followed by Dunnett's test (n=8).

FIG. 5. PPARα agonists and anandamide synergistically inhibit pain. a and b, Anandamide and PEA (0.1 μg/paw of each) synergistically inhibit early (a) and late (b) phase formalin-evoked nociception in Swiss mice (n=8). c and d, Mimicry of this synergism by GW7647 and AEA (0.1 μg/paw each) during the early (c) and late phases (d). (n=10). a-d, The CB1 antagonist SR141716 (SR1; 0.1 mg kg⁻¹, i.v., 10 min before formalin) prevents the effects of anandamide plus either PEA and GW7647 (0.1 μg/paw each). **, P<0.01, ANOVA followed by Dunnett's test (n=8-10).

FIG. 6. PPARα agonists suppress hyperalgesic responses in neuropathic mice. Effects of subcutaneous (s.c.) administration of vehicle (V) or PEA (P, 30 mg-kg⁻¹) on (A) mechanical and (B) thermal withdrawal latencies on day 7 and 14 after ligation of the left sciatic nerve in Swiss mice; BL: baseline responses before surgery (n=8). (C), Dose-dependent effects of s.c. PEA administration on mechanical withdrawal latencies on day 14 after surgery (n=6). Effects of vehicle (V) or GW7647 (GW, 30 mg-kg⁻¹, s.c.) on (D) mechanical and (E) thermal withdrawal latencies on day 7 and 14 after surgery (n=6). (F) Effects of gabapentin (GP, 60 mg-kg⁻¹, s.c.) on mechanical withdrawal latencies on day 14 after surgery (n=6). The drug produced similar effects on thermal withdrawal latency (data not shown). (G) Effects of vehicle (V) or PEA (30 mg-kg⁻¹, s.c, once daily for two weeks) on mechanical withdrawal latencies on day 7 and 14 after surgery (n=8). (H) Immunoblot quantification of PKA-IIα regulatory subunit phosphorylation on Ser⁹⁶ and PKAα catalytic subunit in sham-operated (SH, open bars) or neuropathic (closed bars) Swiss mice treated with vehicle (V) or PEA (30 mg-kg⁻¹, s.c, once daily, for two weeks) (n=3-4). (** P<0.01; ***P<0.001) vs. V; (# P<0.05; ## P<0.01) vs BL; means ±SEM.

FIG. 7. PPARα agonists suppress inflammatory hyperalgesia. Effects of subcutaneous (s.c.) administration of vehicle (V), (A) GW7647 (GW, 30 mg-kg⁻¹), or (B) PEA (P, 30 mg-kg⁻¹) on mechanical withdrawal latencies on days 7 and 14 after adjuvant injection in Swiss mice; BL: baseline response before adjuvant injection (n=8). Effects of s.c. administration of vehicle (V), (C) GW7647 (GW, 30 mg-kg⁻¹), or (D) PEA (P, 30 mg-kg⁻¹) on mechanical withdrawal latencies on day 3 after injection of carrageenan (n=8). (E) Effects of vehicle (V), GW7647 (30 mg-kg⁻¹, s.c.), or PEA (30 mg-kg⁻¹, s.c.) on paw edema in mice treated for 3 days with carrageenan; UN: paw weight in animals not treated with carrageenan (n=6-7). Effects of vehicle (V), PEA or GW7647 (each at 50 μg, i.pl.) on (F) first-phase (I) and second-phase (I) formalin-evoked pain behavior, and (G) paw weight measured 45 min after formalin injection in wild-type C57BL6 mice. UN: paw weight in animals not treated with formalin (n=5). (* P<0.05; ** P<0.01; ***P<0.001) vs. V; (# P<0.05; ## P<0.01) vs BL; means ±SEM.

FIG. 8. Effects of two structurally unrelated CB₂ receptor antagonists, SR144528 and AM630, on PPARα induced antinociception. a, b, Effects of (a) PEA (50 μg, i.pl.), (b) GW7647 (50 μg, i.pl.), or (c) Wy-14643 (WY, 100 μg, i.pl.), alone or in combination with SR144528 (SR2, 1 mg-kg⁻¹, i.v.) on formalin-evoked pain behavior in Swiss mice. d, Effects of PEA (50 μg, i.pl.), alone or in combination with AM630 (2 mg-kg⁻¹, i.p.) on formalin-evoked pain behavior in Swiss mice (n=6-10). *, P<0.05, **, P<0.01; means ±SEM.

FIG. 9. Effects of vehicle (open bars) or PEA (closed bars, 10 μM) on PPARα activation in the presence or absence of the CB₂ antagonist SR144528 (0-5 μM). Data are expressed as fold induction of luciferase activity (n=4). ***, P<0.001; means ±SEM.

FIG. 10. Distribution of PEA in (A) liver, (B) brain and (C) spinal cord following subcutaneous administration of vehicle (V) or PEA (15 or 30 mg-kg⁻¹) to Swiss mice, once daily for 5 consecutive days (n=7). Mice were sacrificed 30 min after the 5^{th} injection, and PEA levels were measured in tissue extracts by liquid chromatography/tandem mass spectrometry (n=6). *, P<0.05, **, P<0.01; means ±SEM.

### DETAILED DESCRIPTION OF THE INVENTION

It has been advantageously discovered that:

(1) OEA selectively engages with high affinity the peroxisome proliferator-activating receptor alpha (PPARα).

(2) The nuclear receptor PPARα is also the molecular target for the effects of palmitoylethanolamide on pain and inflammation. Palmitoylethanolamide and the PPARα agonists GW-7647 and WY-14643 can produce acute analgesia independently of anti-inflammation in a dose dependent manner.

(3) Non-cannabinoid PPARα agonists display synergism with the CB1 endocannabinoid agonists (e.g., anandamide) in formalin-evoked pain models of nociception.

(4) The control of pain initiation by the above PPARα agonists depends on the presence of PPARα insofar as mutant mice, lacking this receptor displayed no response to these drugs.

(5) Importantly, geldanamycin blocked PEA and GW7647 analgesia in the formalin test, indicating a modulatory role of heat shock protein 90 downstream of PPARα activation; and

(6). The anti-inflammatory actions of PEA were abolished in PPAR null mice in carrageenan and phorbol myristic acid induced inflammation.

These results indicate that PPARα agonists, including OEA-like modulators, are particularly useful in the treatment of pain, including neuropathic pain. Further the data show a synergism between PPARα agonists and cannabinoid receptor agonists.

### DEFINITIONS

Each publication, patent application, patent, and other reference cited herein is incorporated by reference in its entirety to the extent that it is not inconsistent with the present disclosure.

It is noted here that as used in this specification and the appended claims, the singular forms “an”, “an,” and “the” include plural reference unless the context clearly dictates otherwise.

Unless defined otherwise, all technical and scientific terms used herein have the meaning commonly understood by a person skilled in the art to which this invention belongs.

The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

Where substituent groups are specified by their conventional chemical formulae, written from left to right, they equally encompass the chemically identical substituents which would result from writing the structure from right to left, e.g., —CH₂O— is intended to also recite —OCH₂—.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. The term “pharmaceutical composition” indicates a composition suitable for pharmaceutical use in a subject, including an animal or human. A pharmaceutical composition generally comprises an effective amount of an active agent and a pharmaceutically acceptable carrier.

Compounds of the invention may contain one or more asymmetric centers and can thus occur as racemates and racemic mixtures, single enantiomers, diastereomeric mixtures and individual diastereomers. The present invention is meant to comprehend all such isomeric forms of the inventive compounds.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed by the inventive formulas.

Compounds of the invention include the diastereoisomers of pairs of enantiomers. Diastereomers for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of an inventive compound may be obtained by stereospecific synthesis using optically pure starting materials or reagents of known configuration

As used herein, the term “heteroatom” is meant to include oxygen (O), nitrogen (N), sulfur (S) and silicon (Si).

“Alkanol,” as used herein, refers to a saturated or unsaturated, substituted or unsubstituted, branched or unbranched alkyl group having a hydroxyl substituent, or a substituent derivable from a hydroxyl moiety, e.g., ether, ester. The alkanol is preferably also substituted with a nitrogen-, sulfur-, or oxygen-bearing substituent that is included in bond Z (Formula I), between the “fatty acid” and the alkanol.

“Fatty acid,” as used herein, refers to a saturated or unsaturated substituted or unsubstituted, branched or unbranched alkyl group having a carboxyl substituent. Preferred fatty acids are C₄-C₂₂ acids.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH≡N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Oleoylethanolamide (OEA) refers to a natural lipid of the following structure:

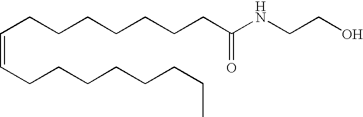

An OEA-like compound includes, but is not limited to, fatty acid alkanolamides, fatty acid ethanolamide compounds, and their analogs and homologues which modulate the PPARα receptor. Exemplary OEA-like compounds are compounds of formula I or Formula VI which modulate the PPARα receptor. OEA-like compounds include agonists and antagonists of the PPARα receptor. OEA-like compounds which selectively modulate the PPARα receptor are preferred. Particularly preferred OEA-like modulators have a selective affinity of at least 10-fold, 50-fold or 100-fold greater for PPARα than for PPARβ or PPARγ. Such preferred OEA-like compound are particularly preferred if they produce a half-maximal effect on the PPARα receptor under physiological conditions at a concentration of 10 micromolar or less, 1 micromolar or less, 100 nanomolar or less, 10 nanomolar or less, or 1 nanomolar or less, or from 1 micromolar to 1.0 nanomolar, or less. Such OEA-like compounds can include, but are not limited to, fatty acid alkanolamides, their homologues and analogues. Particularly preferred OEA-like compounds are also selective for the PPARα receptor over a cannabinoid receptor. Such compounds include those compounds whose affinity for the PPARα receptor is at least 5-fold, 10-fold, or 50-fold greater than that for a cannabinoid receptor (e.g., CB₁ or CB₂ receptor). OEA is an example of a preferred OEA-like compound.

An OEA-like modulator or OEA like agonist is a PPARα agonist having a selective affinity for the PPARα receptor at least 5-fold greater (e.g., having a concentration which produces a half-maximal effect which is at least 5-fold lower) than for either or both PPARβ or PPARγ as measured under comparable bioassay conditions in vivo or in vitro or in any bioassay as described herein. Particularly preferred OEA-like modulators have a selective affinity of at least 5-fold, 10-fold, 50-fold or 100-fold greater for PPARα than for PPARβ or PPARγ. Such preferred OEA-like compounds are particularly preferred if they produce a half-maximal effect on the PPARα receptor under physiological conditions at a concentration of 1 micromolar or less, 100 nanomolar or less, 10 nanomolar or less, or 1 nanomolar or less, or from 1 micromolar to 1.0 nanomolar, or less. Such OEA-like compounds can include, but are not limited to, fatty acid alkanolamides, their homologues and their analogues. Also particularly preferred are OEA and compounds of Formula I or Formula VI. In other embodiments, the OEA-like modulator is a specific high affinity agonist of PPARα which is not a fatty acid alkanolamide or a homolog thereof and is not a compound of Formula I or Formula VI. Particularly preferred OEA-like modulators are selective for the PPARα receptor over a cannabinoid receptor. Such modulators include compounds whose affinity for the PPARα receptor is at least 5-fold, 10-fold, or 50-fold greater than that for a cannabinoid receptor (e.g., CB₁ or CB₂ receptor).

In the formulas herein, “Me” represents the methyl group.

The term “modulate” means to induce any change including increasing or decreasing. (e.g., a modulator of fatty acid oxidation increases or decreases the rate of fatty oxidation. A modulator of a receptor includes both agonists and antagonists of the receptor.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995). Preferred pharmaceutical carriers depend upon the intended mode of administration of the active agent. Typical modes of administration are described below.

The term “effective amount” means a dosage sufficient to produce a desired result on health. The desired result may comprise a subjective or objective improvement in the recipient of the dosage. A subjective improvement may be, for instance, decreased sensation of pain (e.g., noninflammatory pain, neuropathic pain). An objective improvement may be, for instance, an increased ability to move or use (e.g., place weight upon) an affected limb or a longer period of uninterrupted sleep.

A “prophylactic treatment” is a treatment administered to a subject who does not have pain, wherein the treatment is administered for the purpose of decreasing the risk of developing a noninflammatory or neuropathic pain.

A “therapeutic treatment” is a treatment administered to a subject who exhibits signs or symptoms of noninflammatory neuropathic pain, wherein treatment is administered for the purpose of diminishing or eliminating those pathological signs or symptoms. A “therapeutically effective amount” is thus an amount of an agent sufficient to reduce such signs and/or symptoms or to prevent their progression.

An activation assay is an assay that provides an assessment of the in vivo activation of transcription activators in response to extracellular stimuli. The assessment may be provided by measurement of reporter gene activation, measurement of PPARα mRNA levels, or proliferation of cells transfected with PPARα. It includes assays wherein the activation of PPARα that results from PPARα-RXR heterodimer formation that results from binding of a PPARα subtype specific ligand to PPARα.

An agonist is a ligand of a receptor which activates the receptor or causes signal transduction upon binding to the receptor. OEA is an example of a PPARα receptor agonist.

An antagonist is a ligand of a receptor which binds to the receptor but does not appreciably activate the receptor or appreciably cause signal transduction. An antagonist may block the ability of an agonist to bind and activate a receptor or otherwise reduce the activity of the receptor under physiological conditions.

A binding assay is an assay that provides an assessment of ligand binding to a receptor (e.g., PPARα, PPARβ, or PPARγ receptors). For instance, the assessment may be provided by measurement of displacement of radioactively labeled PPARα ligand, of electrophoretic mobility shifts, measurement of immunoprecipitation of PPARα, PPARβ, or PPARγ by antibodies. The assessment may be accomplished through high throughput screening. A “specific” binder or binding of PPARα refers to a compound or binding interaction that has at least 5 fold greater affinity (e.g., as measured by EC₅₀'s or IC₅₀'s) for PPARα than for PPARγ or for PPARβ. Binding is not determinative that a ligand is an agonist or an antagonist.

A peroxisome proliferator activated receptor (PPAR) is a member of a family of nuclear receptors, distinguished in α, β, and γ subtypes as described herein.

A specific or selective PPAR activator is a compound that preferentially binds and activates one PPAR subtype over another. For example, a specific activator of PPARα is OEA. In all embodiments setting forth a class of PPARα agonists to be used according to the invention, in a further one of each such embodiments, the PPARα agonist is not PEA. In another further one of such embodiments, the PPARα agonist is not a CB2 cannabinoid agonist or a CB1 cannabinoid agonist. In some embodiments for each of the aspects of the invention, the PPARα agonist to be used according to the invention is a specific PPARα agonist.

Fatty acid amide hydrolase is the enzyme primarily responsible for the hydrolysis of anandamide in vivo. It also is responsible for the hydrolysis of OEA in vivo. Inhibitors of the enzyme are well known to one of ordinary skill in the art.

A specific or selective binder is a compound that preferentially binds one receptor type over another. For example, a specific binder of PPARα over PPARβ is OEA.

“Neuropathic pain” is pain caused by a primary lesion or dysfunction of the nervous system. Such pain is chronic and involves a maintained abnormal state of increased pain sensation, in which a reduction of pain threshold and the like are continued, due to persistent functional abnormalities ensuing from an injury or degeneration of a nerve, plexus or perineural soft tissue. Such injury or degeneration may be caused by wound, compression, infection, cancer, ischemia, or a metabolic or nutritional disorder such as diabetes mellitus. Neuropathic pain includes, but is not limited to, neuropathic allodynia wherein a pain sensation is induced by mechanical, thermal or another stimulus that does not normally provoke pain, neuropathic hyperalgesia wherein an excessive pain occurs in response to a stimulus that is normally less painful than experienced. Examples of neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. The neuropathic pain includes the pain caused by either central or peripheral nerve damage. And it includes the pain caused by either mononeuropathy or polyneuropathy (e.g., familial amyloid polyneuropathy). As compared to inflammatory pain, neuropathic pain is relatively resistant to therapy with nonsteroidal anti-inflammatory agents and opioid substances (e.g, morphine).

Neuropathic pain may be bilateral in mirror image sites, or may be distributed approximately according to the innervation of the injured nerve, it may persist for months or years, and be experienced as a burning, stabbing, shooting, throbbing, piercing electric shock, or other unpleasant sensation.

The subject species to which the treatments can be given according to the invention are mammals, and include, but are not limited to, humans, primates, rodents, rats, mice, rabbits, horses, dogs and cats.

The vanilloid receptor (VR1), which was recently cloned by Caterina et al., Nature 389, 816-824 (1997) is a capsaicin-sensitive, heat-gated, non-selective cation channel. Methods for screening compounds for vanilloid receptor activity are well known in the art. See U.S. Patent Application Publication No. 20040063786, published Apr. 1, 2004. See U.S. Patent Application Publication No. 20020019444, published Feb. 14, 2002.

### Compounds for Use According to the Invention

Compounds for use according to the present invention (e.g., OEA-like compounds, OEA-like modulators, FAAH inhibitors) may possess asymmetric carbon atoms (optical centers) or double bonds; the racemates, diastereomers, geometric isomers and individual isomers are all intended to be encompassed within the scope of the present invention.

Such compounds for use according to the invention may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of such a compound for use according to the invention may be obtained by stereospecific synthesis using optically pure starting materials of known configuration.

The compounds for use according to the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention.

The compounds may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counterion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

The invention also encompasses prodrugs of OEA-like compounds, OEA-like modulators, and FAAH inhibitors which on administration undergo chemical conversion by metabolic processes before becoming active pharmacological substances. In general, such prodrugs will be derivatives of the present compounds that are readily convertible in vivo into a functional compound of the invention. Conventional procedures for the selection and preparation of suitable prodrug derivatives are described, for example, in “Design of Prodrugs”, ed. H. Bundgaard, Elsevier, 1985. The invention also encompasses active metabolites of the present compounds.

### A. Fatty Acid Alkanolamide Compounds, Homologs, and Analogs.

OEA-like compounds and OEA-like modulators for use according to the invention include, but are not limited to fatty acid ethanolamide compounds, and their homologues. A variety of OEA-like compounds and OEA-like modulators are contemplated. These compounds include compounds having the following general formula:

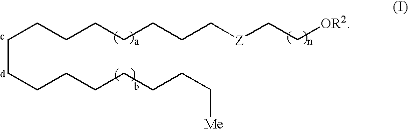

In this formula, n is any number from 0 to 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of unsubstituted or unsubstituted alkyl, hydrogen, substituted or unsubstituted C₁-C₆ alkyl, substituted or unsubstituted lower (C₁-C₆) acyl, homoalkyl, and aryl. Up to eight hydrogen atoms of the compound may also be substituted by methyl group or a double bond. In addition, the molecular bond between carbons c and d may be unsaturated or saturated. In some embodiments, the fatty acid ethanolamide of the above formula is a naturally occurring compound. In some preferred embodiments, the alkyl substituents are each homoalkyl.

OEA-like compounds and OEA-like modulators of the invention also include compounds of the following formula:

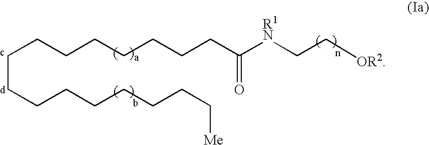

In one embodiment, the compounds of Formula Ia have n from 0 to 5; and a sum of a and b that is from 0 to 4; and members R¹ and R² independently selected from the group consisting of hydrogen, substituted or unsubstituted C₁-C₆ alkyl, lower substituted or unsubstituted (C₁-C₆) acyl, homoalkyl, and substituted or unsubstituted aryl. In this embodiment, up to eight hydrogen atoms of the fatty acid portion and alkanolamine (e.g., ethanolamine) portion of compounds of the above formula may also be substituted by methyl or a double bond if adjacent carbons. In addition, the molecular bond between carbons c and d may be unsaturated or saturated. In some embodiments with acyl groups, the acyl groups may be the propionic, acetic, or butyric acids and attached via an ester linkage as R² or an amide linkage as R¹. In some embodiments, a H atom attached to a carbon atom of a compound of the above formula is replaced with a halogen atom, preferably a Cl atom or a F atom.

In another embodiment, the above compounds particularly include those in which the fatty acid moiety comprises oleic acid, elaidic acid, or palmitic acid. Such compounds include oleoylethanolamide, elaidylethanolamide and palmitoylethanolamide.

In still another embodiment, the compounds of Formula Ia have n from 1 to 3; and a sum of a and b that is from 1 to 3; and members R¹ and R² independently selected from the group consisting of hydrogen, substituted or unsubstituted C₁-C₆ alkyl, and lower substituted or unsubstituted (C₁-C₆) acyl. In this embodiment, up to four hydrogen atoms of the fatty acid portion and alkanolamine (e.g., ethanolamine) portion of compounds of the above formula may also be substituted by methyl or a double bond. In addition, the molecular bond between carbons c and d may be unsaturated or saturated. In a further embodiment, the molecular bond between carbons c and d is unsaturated and no other hydrogen atoms are substituted. In a still further embodiment thereof, the members R¹ and R² are independently selected from the group consisting of hydrogen, substituted or unsubstituted C₁-C₃ alkyl, and substituted or unsubstituted lower (C₁-C₃) acyl.

Exemplary compounds provide mono-methyl substituted compounds, including ethanolamides, of Formula Ia. Such compounds include:

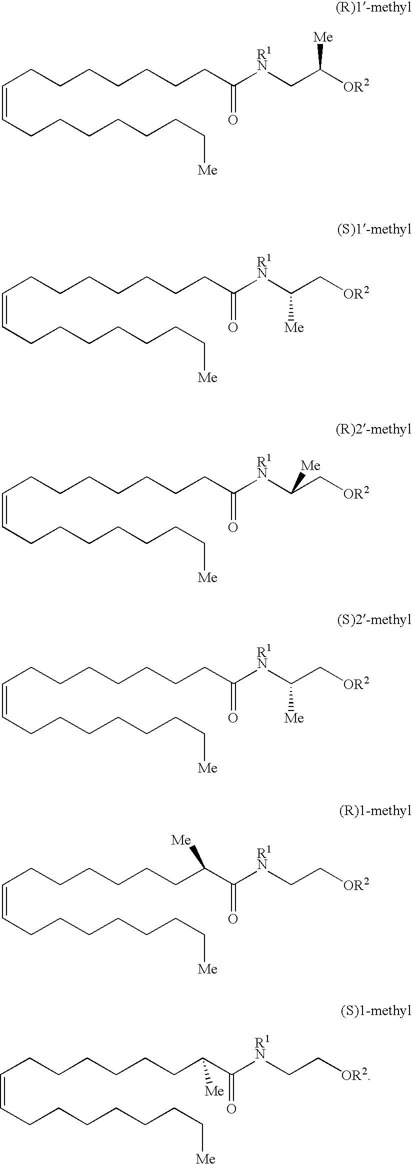

The methyl substituted compounds of the above formula include particularly those compounds where R¹ and R² are both H: (R)1′-methyloleoylethanolamide, S(1′)-methyloleoylethanolamide, (R)2′-methyloleoylethanolamide, (S)2′-methyloleoylethanolamide, (R)1-methyloleoylethanolamide, and (S)1-methyloleoylethanolamide.

### Reverse OEA-Like Compounds.

OEA-like compounds and OEA-like modulators for use according to the invention also include a variety of analogs of OEA. These compounds include reverse OEA compounds of the general formula:

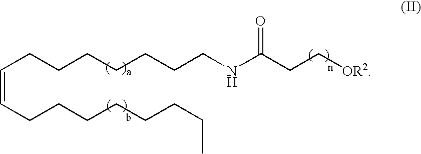

In some embodiments, the invention provides compounds of Formula II. Exemplary the compounds of Formula II have n from 1 to 5, and a sum of a and b from 0 to 4. In this embodiment, the member R² is selected from the group consisting of hydrogen, substituted or unsubstituted C₁-C₆ alkyl; substituted or unsubstituted lower (C₁-C₆) acyl, homoalkyl, and aryl. In addition, up to four hydrogen atoms of either or both the fatty acid portion and alkanolamine (e.g., ethanolamine) portion of compounds of the above formula may also be substituted by methyl or a double bond.

Exemplary compounds of formula II include those compounds where the alkanolamine portion is ethanolamine, compounds where R² is H, and compounds where a and b are each 1, and compounds where n is 1.

One embodiment of a compound according to Formula II is

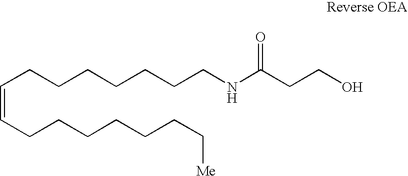

In another embodiment, the compounds of Formula II have n from 1 to 5 and a sum of a and b from 1 to 3. In this embodiment, the member R² is selected from the group consisting of hydrogen, substituted or unsubstituted C₁-C₆ alkyl, and substituted or unsubstituted lower (C₁-C₆) acyl. In addition, up to four hydrogen atoms of either or both the fatty acid portion and alkanolamine (e.g., ethanolamine) portion of compounds of the above formula may also be substituted by methyl or a double bond.

### Oleoylalkanol Ester Compounds.

OEA-like compounds and OEA-like modulators of the invention also include oleoylalkanol esters of the general formula:

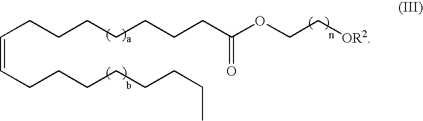

In some embodiments, the compounds of Formula III, have n from 1 to 5; and the sum of a and b from 0 to 4. The member R² is selected from the group consisting of hydrogen, substituted or unsubstituted C₁-C₆ alkyl, lower (C₁-C₆) acyl, homoalkyl, and aryl. Up to four hydrogen atoms of either or both the fatty acid portion and alkanol (e.g., ethanol) portion of compounds of the above formula may also be substituted by methyl or a double bond.

In some embodiments, the compounds of Formula III, have n from 1 to 3; and the sum of a and b from 1 to 3. The member R² is selected from the group consisting of hydrogen, substituted or unsubstituted C₁-C₆ alkyl, and substituted or unsubstituted lower (C₁-C₆) acyl. Up to four hydrogen atoms of the fatty acid portion and alkanol (e.g., ethanol) portion of compounds of the above formula may also be substituted by methyl or a double bond.

Compounds of Formula III include those compounds where R² is H, compounds where a and b are each 1, and compounds where n is 1. Examples of compounds according to Formula III include the oleoyldiethanol ester:

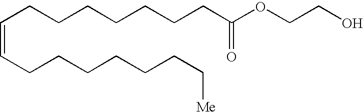

Compounds of Formula III also include mono-methyl substituted oleoyl ethanol esters such as the (R or S)-2′-methyloleoylethanolesters; the (R or S)-1′-methyloleoylethanolesters; and the (R or S))-1′-methyloleoylethanolesters; respectively:

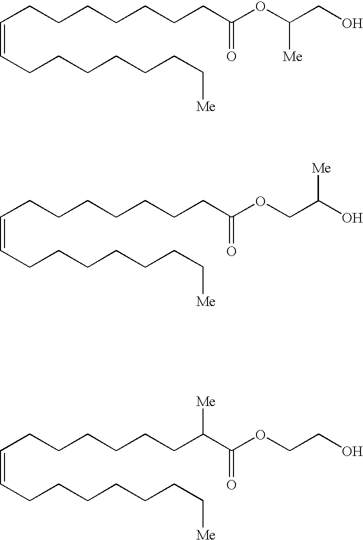

### Oleoyl Alkanol Ethers

OEA-like compounds and OEA-like modulators of the invention also include oleoylalkanol ethers according to the general formula:

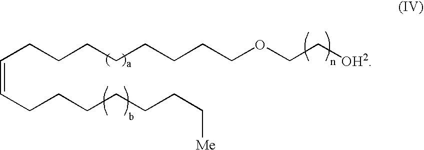

In some embodiments, the compounds of Formula IV, have an n from 1 to 5 and a sum of a and b that can be from 0 to 4. The member R² is selected from the group consisting of hydrogen, substituted or unsubstituted C₁-C₆ alkyl, substituted or unsubstituted lower (C₁-C₆) acyl, alkyl, and substituted and unsubstituted aryl. Up to four hydrogen atoms of either or both the fatty acid portion and alkanol (e.g., ethanol) portion of compounds of the above formula may also be substituted by methyl or a double bond.

In other embodiments, the compounds of Formula IV, have n from 1 to 3; and the sum of a and b can be from 1 to 3. The member R² is selected from the group consisting of hydrogen, substituted or unsubstituted C₁-C₆ alkyl, and substituted or unsubstituted lower (C₁-C₆) acyl. Up to four hydrogen atoms of either or both the fatty acid portion and alkanol (e.g., ethanol) portion of compounds of the above formula may also be substituted by methyl or a double bond.

Compounds of Formula IV include those compounds where R² is H, compounds where a and b are each 1, and compounds where n is 1. Examples of compounds according to Formula IV include the following (R or S) 1′-oleoylethanol ethers and (R or S)-2′-oleoylethanol ethers:

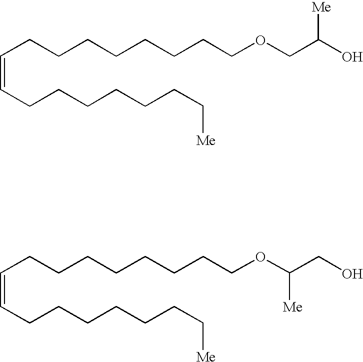

### Fatty Acid Alkanolamide Analogs Having Polar Head Variants.

OEA-like compounds and OEA-like modulators for use according to the invention include compounds having a variety of polar head analogs of OEA. These compounds include compounds having a fatty acid moiety of the general formula:

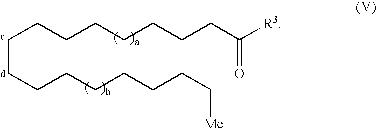

In some embodiments, the compounds of Formula V have a sum of a and b that can be from 0 to 4. In other embodiments, the sum of a and b is from 1 to 3. In these embodiments, up to four hydrogen atoms of the compounds of the above formula may also be substituted by methyl or a double bond. In addition, the molecular bond between carbons c and d may be unsaturated or saturated. A particularly preferred embodiment is that of the oleic acid fatty acid moiety:

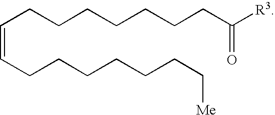

The R³ group of the above structures may be selected from any of the following:

HO—(CH₂)_{z}—NH— wherein z is from 1 to 5, and the alkyl portion thereof is an unbranched methylene chain. For example:

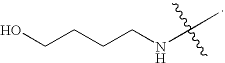

H₂N—(CH₂)_{z}—NH— wherein z is from 1 to 5, and the alkyl portion thereof is an unbranched methylene chain. For example:

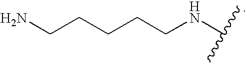

HO—(CH₂)ₓ—NH— wherein x is from 1 to 8, and the alkyl portion thereof may be branched or cyclic. For example,

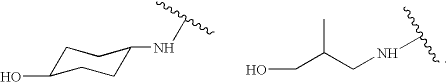

Additional polar head groups for R³ include, for instance, compounds having furan, dihydrofuran and tetrahydrofuran functional groups:

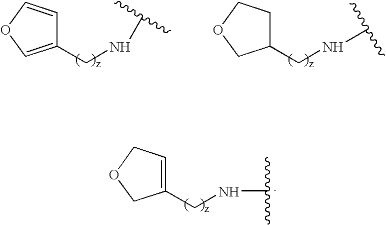

### In the above structures, z can be from 1 to 5.

Such compounds for use according to the invention include, for instance, those having R³ polar head groups based upon pyrole, pyrrolidine, and pyrroline rings:

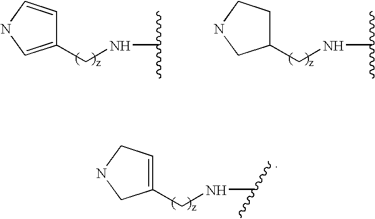

### In the compounds of the above structures, z can be from 1 to 5.

Other exemplary polar head groups include a variety of imidazole and oxazoles, for example:

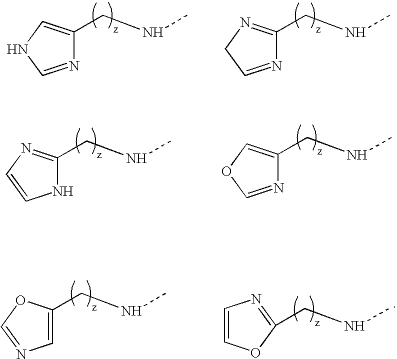

### In the compounds of the above structures, z can be from 1 to 5.

Oxazolpyridine polar head groups are also exemplary:

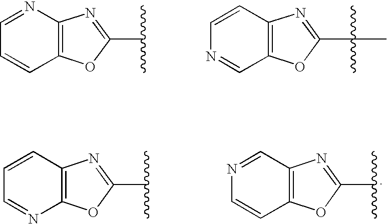

### Fatty Acid Alkanolamide Analogs Having Apolar Tail Variants.

OEA-like compound and OEA-like modulators for use according to the invention include a variety of alkanolamide and ethanolamide compounds having a variety of flexible apolar tails. These compounds include compounds of the following formulas in which R represents an ethanolamine moiety, an alkanolamine moiety, or a stable analog thereof. In the case of ethanolamine, the ethanolamine moiety is attached preferably via the ethanolamine nitrogen rather than the ethanolamine oxygen.

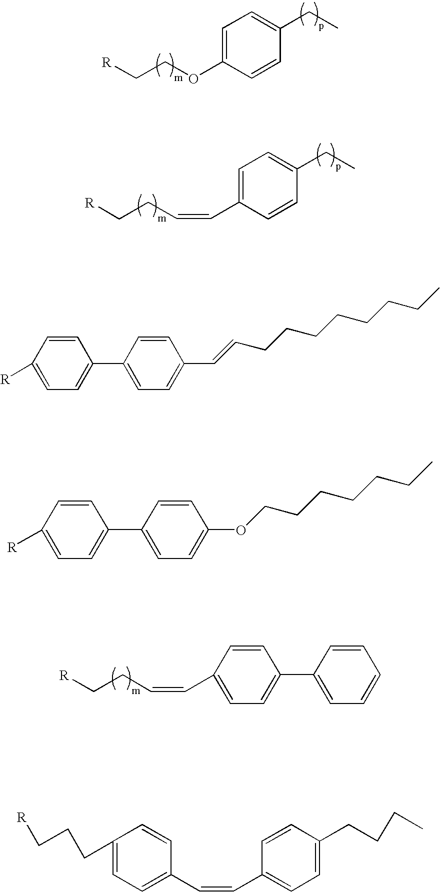

### In the above structures, m is from 1 to 9 and p is independently from 1 to 5.

An exemplary compound for use is:

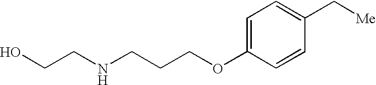

Another exemplary compound for use is an ethanolamine analog with an apolar tail of the following structural formula:

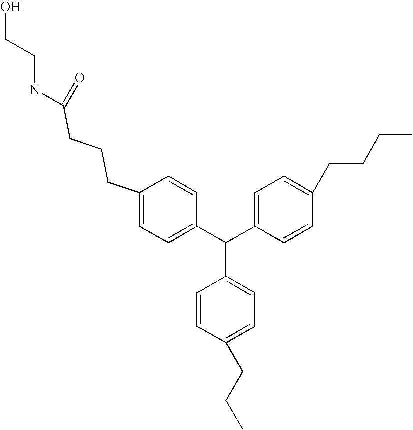

OEA-like compound and OEA-like modulators of the invention of the invention include those disclosed in U.S. patent application Ser. No. 10/112,509 filed Mar. 27, 2002, assigned to the same assignee as the present application, which is incorporated herein by reference. In other embodiments, the fatty acid moiety of the fatty acid alkanolamide or ethanolamide compound, homologue, or analog may be saturated or unsaturated, and if unsaturated may be monounsaturated or polyunsaturated.

In some embodiments, the fatty acid moiety of the fatty acid alkanolamide compound, homologue, or analog is a fatty acid selected from the group consisting of oleic acid, palmitic acid, elaidic acid, palmitoleic acid, linoleic acid, α-linolenic acid, and γ-linolenic acid. In certain embodiments, the fatty acid moieties have from twelve to 20 carbon atoms.

Other embodiments are provided by varying the hydroxyalkylamide moiety of the fatty acid amide compound, homologue or analog. These embodiments include the introduction of a substituted or unsubstituted lower (C₁-C₃) alkyl group on the hydroxyl group of an alkanolamide or ethanolamide moiety so as to form the corresponding lower alkyl ether. In another embodiment, the hydroxy group of the alkanolamide or ethanolamide moiety is bound to a carboxylate group of a C₂ to C₆ substituted or unsubstituted alkyl carboxylic acid to form the corresponding ester of the fatty acid ethanolamide. Such embodiments include fatty acid alkanolamide and fatty acid ethanolamides in ester linkage to organic carboxylic acids such as acetic acid, propionic acid, and butanoic acid. In one embodiment, the fatty acid alkanolamide is oleoylalkanolamide. In a further embodiment, the fatty acid alkanolamide is oleoylethanolamide.

In still another embodiment, the fatty acid ethanolamide compound, homologue, or analog further comprises a substituted or unsubstituted lower alkyl (C₁-C₃) group covalently bound to the nitrogen atom of the fatty acid ethanolamide.

In still another embodiment, the compound of the invention is fatty acid alkanolamide compound or homologue satisfying the following formula VI:

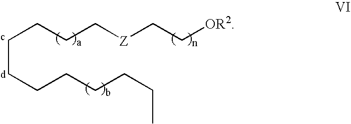

In this formula, n is any number from 0 to 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of unsubstituted or unsubstituted alkyl, hydrogen, substituted or unsubstituted C₁-C₆ alkyl, substituted or unsubstituted lower (C₁-C₆) acyl, homoalkyl, and aryl. Up to six hydrogen atoms of the compound may also be substituted by methyl group or a double bond. In addition, the molecular bond between carbons c and d may be unsaturated or saturated. In some embodiments, the fatty acid ethanolamide of the above formula is a naturally occurring compound. In some preferred embodiments, the alkyl substitutents are each homoalkyl, or its pharmaceutically acceptable salt. Further embodiments of the compounds of Formula VI have substituents as set forth for compounds of Formula I above. In some embodiments, a H atom attached to a carbon atom of a compound of the above formula is replaced with a halogen atom, preferably a Cl atom or a F atom.

### Synthesis of Fatty Acid Alkanolamides

Compounds useful in practicing the present invention can be readily synthesized and purified using methods recognized in the art. In an exemplary synthetic scheme (Scheme 1) a carboxylic acid and an aminoalcohol (or an O-protected derivative thereof) are reacted in a the presence of a dehydrating agent, e.g., dicyclohexylcarbodiimide, in an appropriate solvent. The fatty acid alkanol amide is isolated by methods such as extraction, crystallization, precipitation, chromatography and the like. If the final product is the O-protected adduct, it is deprotected, typically by an art-recognized method, to afford a fatty acid adduct having a free hydroxyl group.

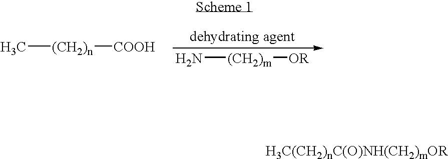

Those of skill in the art will recognize that many variants on the scheme set forth above are available. For example, an activated derivative, e.g, acyl halide, active ester, of the acid can be used. Similarly, a glycol (preferably mono O-protected) can be substituted for the amino alcohol, resulting in an ester linkage between the two constituents of the molecule.

Reverse esters and reverse amides can also be readily synthesized by art-recognized methods. For example, a hydroxycarboxylic acid is reacted with an amine or hydroxy derivative of a long chain alkyl (i.e., C₄-C₂₂) in the presence of a dehydrating agent. In certain reaction pathways, it is desirable to protect the hydroxyl moiety of the hydroxycarboxylic acid.

Ethers and mercaptans can be prepared by methods well-known to those of skill in the art, e.g., Williamson synthesis. For example, a long chain alkyl alcohol or thiol is deprotonated by a base, e.g, NaH, and a reactive alcohol derivative, e.g., a halo, tosyl, mesyl alcohol, or a protected derivative thereof is reacted with the resulting anion to form the ester or mercaptan.

The above-recited methods and variations thereof can be found in, for example, RECENT DEVELOPMENTS IN THE Synthesis OF FATTY ACID DERIVATIVES, Knothe G, ed., Amer. Oil Chemists Society 1999; COMPREHENSIVE NATURAL PRODUCTS CHEMISTRY AND OTHER SECONDARY METABOLITES INCLUDING FATTY ACIDS AND THEIR DERIVATIVES, Nakanishi K, ed., Pergamon Press, 1999; ORGANIC SYNTHESIS COLLECTED VOLUMES I-V, John Wiley and Sons; COMPENDIUM OF ORGANIC SYNTHETIC METHODS, Volumes 1-6, Wiley Interscience 1984; ORGANIC FUNCTIONAL GROUP PREPARATION, Volumes I-III, Academic Press Ltd. 1983; Greene T, PROTECTING GROUPS IN ORGANIC SYNTHESIS, 2d ed., Wiley Interscience 1991.

### Other PPARα Agonists for Use According to the Invention.

In addition, the PPARα agonists need not be an OEA-like compound (e.g., OEA, fatty acid amide or homolog thereof). In some embodiments, the OEA-like modulator is a compound such as taught in U.S. Pat. No. 6,200,998 (hereby incorporated by reference) that are PPARα activators. This reference teaches PPAR agonist compounds of the general formula:

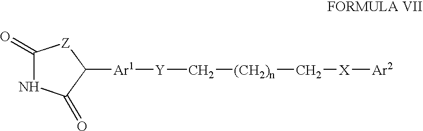

In the above formula, Ar¹ is (1) arylene or (2) heteroarylene, wherein arylene and heteroarylene are optionally substituted with from 1 to 4 groups selected from R^{a} (defined below); Ar² is (1) ortho-substituted aryl or (2) ortho-substituted heteroaryl, wherein said ortho substituent is selected from R (defined below); and aryl and heteroaryl are optionally further substituted with from 1-4 groups independently selected from R^{a}; X and Y are independently O, S, N—R^{b} (defined below), or CH₂; Z is O or S; n is 0 to 3; R is (1) C₃₋₁₀ alkyl optionally substituted with 1-4 groups selected from halo and C₃₋₆ cycloalkyl, (2) C₃₋₁₀ alkenyl, or (3) C₃₋₈ cycloalkyl; R^{a} is (1) C₁₋₁₅ alkanoyl, (2) C₁₋₁₅ alkyl, (3) C₂₋₁₅ alkenyl, (4) C₂₋₁₅ alkynyl, (5) halo, (6) OR^{b}, (7) aryl, or (8) heteroaryl, wherein said alkyl, alkenyl, alkynyl, and alkanoyl are optionally substituted with from 1-5 groups selected from R^{c} (defined below), and said aryl and heteroaryl optionally substituted with 1 to 5 groups selected from R^{d} (defined below); R^{b} is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl, wherein alkyl, alkenyl, alkynyl are optionally substituted with one to four substituents independently selected from R^{c}, and cycloalkyl, aryl and heteroaryl are optionally substituted with one to four substituents independently selected from R^{d}; or R^{c} is (1) halo, (2) aryl, (3) heteroaryl, (4) CN, (5) NO₂, (6) OR^{f}; (7) S(O)ₘR^{f}, m=0, 1 or 2, provided that R^{f} (defined below) is not H when m is 1 or 2; (8) NR^{f}R^{f} (9) NR^{f}COR^{f}, (10) NR^{f}CO₂R^{f}, (11) NR^{f}CON(R^{f})₂, (12) NR^{f}SO₂R^{f}, provided that R^{f} is not H, (13) COR^{f}, (14) CO₂R^{f}, (15) CON(R^{f})₂, (16) SO₂N(R^{f})₂, (17) OCON(R^{f})₂, or (18) C₃₋₈ cycloalkyl, wherein said cycloalkyl, aryl and heteroaryl are optionally substituted with 1 to 3 groups of halo or C₁₋₆alkyl; R^{d} is (1) a group selected from R^{c}, (2) C₁₋₁₀ alkyl, (3) C₂₋₁₀ alkenyl, (4) C₂₋₁₀ alkynyl, (5) aryl C₁₋₁₀ alkyl, or (6) heteroaryl C₁₋₁₀ alkyl, wherein alkyl, alkenyl, alkynyl, aryl, heteroaryl are optionally substituted with a group independently selected from R^{e}; R^{e} is (1) halogen, (2) amino, (3) carboxy, (4) C₁₋₄ alkyl, (5) C₁₋₄ alkoxy, (6) hydroxy, (7) aryl, (8) aryl C₁₋₄ alkyl, or (9) aryloxy; R^{f} is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl; wherein alkyl, alkenyl, alkynyl, aryl, heteroaryl, alkanoyl and cycloalkyl are optionally substituted with one to four groups selected from R^{e}.

Also preferred are those PPARα specific activators as taught in U.S. Pat. No. 5,859,051. These activators have the following general formula as set forth in the U.S. Pat. No. 5,589,051:

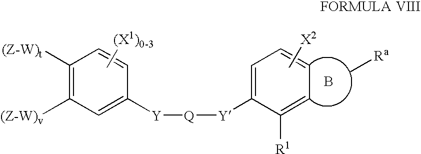

In the embodiments according to Formula VIII, R¹ is selected from a group consisting of: H, C₁₋₁₅ alkyl, C₂₋₁₅ alkenyl, C₂₋₁₅ alkynyl and C₃₋₁₀ cycloalkyl, said alkyl, alkenyl, alkynyl, and cycloalkyl optionally substituted with 1 to 3 groups of R^{a} (defined below); R³ is selected from a group consisting of: H, NHR¹, NHacyl, C₁₋₁₅ alkyl, C₃₋₁₀ cycloalkyl, C₂₋₁₅ alkenyl, C₁₋₁₅ alkoxy, CO₂ alkyl, OH, C₂₋₁₅ alkynyl, C₅₋₁₀ aryl, C₅₋₁₀ heteroaryl said alkyl, cycloalkyl, alkenyl, alkynyl, aryl and heteroaryl optionally substituted with 1 to 3 groups of R^{a}; (Z-W—) is Z-CR⁶R⁷—, Z-CH.═CH—, or:

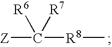

R⁸ is selected from the group consisting of CR⁶R⁷, O, NR⁶, and S(O)_{P}; R⁶ and R⁷ are independently selected from the group consisting of H, C₁₋₆ alkyl; B is selected from the group consisting of: 1) a 5 or 6 membered heterocycle containing 0 to 2 double bonds, and 1 heteroatom selected from the group consisting of O, S and N, the heteroatom being substituted at any position on the five or six membered heterocycle, the heterocycle being optionally unsubstituted or substituted with 1 to 3 groups of R^{a}; 2) a 5 or 6 membered carbocycle containing 0 to 2 double bonds, the carbocycle optionally unsubstituted or substituted with 1 to 3 groups of R^{a} at any position on the five or six membered carbocycle; and 3) a 5 or 6 membered heterocycle containing 0 to 2 double bonds, and 3 heteroatoms selected from the group consisting of O, N, and S, which are substituted at any position on the five or six membered heterocycle, the heterocycle being optionally unsubstituted or substituted with 1 to 3 groups of R^{a}; X¹ and X² are independently selected from a group consisting of: H, OH, C₁₋₁₅ alkyl, C₂₋₁₅ alkenyl, C₂₋₁₅ alkynyl, halo, OR³, ORCF₃, C₅₋₁₀ aryl, C₅₋₁₀ aralkyl, C₅₋₁₀ heteroaryl and C₁₋₁₀ acyl, said alkyl, alkenyl, alkynyl, aryl and heteroaryl optionally substituted with 1 to 3 groups of R^{a}; R^{a} represents a member selected from the group consisting of: halo, acyl, aryl, heteroaryl, CF₃, OCF₃, —O—, CN, NO₂, R³, OR³; SR³, ═N(OR), S(O)R³, SO₂R³, NR³R³, NR³COR³, NR³CO₂R³, NR³CON(R³)₂, NR³SO₂R³, COR³, CO₂R³, CON(R³)₂, SO₂N(R³)₂, OCON(R³)₂ said aryl and heteroaryl optionally substituted with 1 to 3 groups of halo or C₁₋₆ alkyl; Y is selected from the group consisting of: S(O)ₚ, —CH₂—, —C(O)—, —C(O)NH—, —NR—, —O—, —SO₂NH—, —NHSO₂; Y¹ is selected from the group consisting of: O and C; Z is selected from the group consisting of: CO₂R³, R³CO₂R³, CONHSO₂Me, CONHSO₂, CONH₂ and 5-(1H-tetrazole); t and v are independently 0 or 1 such that t+v=1 Q is a saturated or unsaturated straight chain hydrocarbon containing 2-4 carbon atoms and p is 0-2 with the proviso when Z is CO₂R³ and B is a 5 membered heterocycle consisting of O, R³ does not represent methyl.

Additional compounds suitable for practicing the inventive methods include compounds taught in U.S. Pat. No. 5,847,008, U.S. Pat. No. 6,090,836 and U.S. Pat. No. 6,090,839, U.S. Pat. No. 6,160,000 each of which is herein incorporated by reference in its entirety to the extent not inconsistent with the present disclosure.

Additionally a variety of suitable PPAR agonists and activators for screening are taught in U.S. Pat. No. 6,274,608. Aryl and heteroaryl acetic acid and oxyacetic acid compounds are taught for instance in U.S. Pat. No. 6,160,000; substituted 5-aryl-2,4-thiazolidinediones are taught in U.S. Pat. No. 6,200,998; other compounds including PPARα-specific polyunsaturated fatty acids and eicosanoids are known as described in Forman, B M, Chen, J, and Evans R M, PNAS 94:4312-4317 and PCT Patent Publication No. WO 97/36579, published Oct. 9, 1997). The compositions of these publications, which are each herein incorporated by reference in their entirety to the extent not inconsistent with the present disclosure can be screened by the methods provide below to provide the PPARα specific agonists of the invention which are useful in treating neuropathic pain.

In some embodiments, the PPARα agonist is clofibrate or a derivative of clofibrate. Such compounds include, but are not limited to, clofibrate (i.e., 2-(4-chlorophenoxy)-2-methylpropanoic acid, ethyl ester); fenofibrate, (1-methylethyl 2-[4-(4-chlorobenzoyl)phenoxy]-2-methylpropanoate; 2-[4-(4-chlorobenzoyl)phenoxy]-2-methylpropanoic acid, 1-methylethyl ester); bezafibrate (2-[4-[2-[(4-chlorobenzoyl)amino]-ethyl]phenoxy]-2-methyl-propanoic acid, gemfibrozil: 5-(2,5-dimethylphenoxy)-2,2-dimethylpentanoic acid and ciprofibrate.

Other PPARα agonists suitable for use in the methods and compositions of the invention are clofibrate derivative compounds of the following formula or their pharmaceutically acceptable salts:

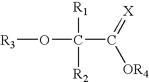

wherein R₁ and R₂ may be the same or different and are each a hydrogen atom or a substituted or unsubstituted alkyl, alkoxy, or phenoxy group, R₃ is a substituted or unsubstituted aryl group phenyl group and X is hydrogen (2H) or oxygen, and R₄ is H or alkyl. In one embodiment, the R₃ aryl group is substituted or unsubstituted phenyl, preferably monosubstituted. In another embodiment, X is O and R₃ is a mono-, di- or tri-substituted phenyl group, bearing one, two or three identical or different substituents for an aryl group and R₁ and R₂ are each, independently, a hydrogen atom or an alkyl group. In a further embodiment, R₃ is a is a mono-, di- or tri-substituted phenyl group, bearing one, two or three identical or different substituents which are one or more of the following, namely halogen atoms and alkyl, alkoxy, aryl, heteroaryl, or hydroxy groups, and R₁ and R₂ are each, independently, a hydrogen atom or an alkyl group, and R₄ is H or alkyl.

Additional PPARα agonists for use according to the invention include:

WY-14,643 (i.e., [4-chloro-6-(2,3-xylidino)-2-pyrimidinylthio]acetic acid)

Fenofibrate (see U.S. Pat. Nos. 5,830,148; 6,074,670; 5,827,536; 5,545,628; 6,277,405 and Casas, F. et al., FEBS Lett., 482(1-2): 71-4 (2000)).

Medium and long chain fatty acids (see U.S. Pat. Nos. 6,008,237; 6,200,998)

Arylthiazolidinedione derivatives (see U.S. Pat. Nos. 6,200,998; 6,008,237)

Propionic acid derivaties (see U.S. Pat. No. 6,306,854)

Pioglitazone (see Smith, U., Int. J. Clin. Pract. Suppl., 121: 13-18 (2001))

Benzafibrate(bezafibrate) (see Yoshikawa et al., Eur. J. Pharmacol., 426(3): 2001-6 (2001); Bonilla, S. et al., J. Physiol. Biochem., 57(1): 1-8 (2001); Pedraza, N., et at., Diabetes, 49(7): 1224-30 (2000).

(−) DRF2725 (i.e., (−)3-[4-[2-(phenoxazin 10-yl)ethoxyl]phenyl]-2-ethoxypropionic acid) (see Lohray, B. B. et al., J. Med. Chem., 44(15): 2675-8 (2001))

BM-17.0744 (see Carroll, R. et al., Physiol. Heart Circ. Physiol., 281(2): H888-94 (2001)) ciprofibrate (see Latruffe, N. et al., Cell Biochem. Biophys., 32 Spring: 213-20 (2000));

Omega-3-fatty acids, including docosahexanoic acid (see Diep, Q. H. et al., Hypertension, 36(5): 851-5 (2000))

Clofibrate (see Mehendale, H. M., Toxicol. Sd., 57(2): 187-90 (2000))

JTT-501 4-[4-[2-(5-methyl-2-phenyl]-4-oxazolyl)ethoxy)benzyl]-3,5-isoxazolidiedione (see Shibata, T. et al., Br. J. Pharmacol., 30(3): 495-504 (2000)

Trichloroacetate, dichloroacetate; DHEA-S dehydroepiandrosterone-3-beta-sulfate (see-Zhou, Y. C. et al, Environ. Health Perspect., 106(Suppl.; 4): 983-988 (1998)

Unsaturated C:18 fatty acids (e.g., arachidonic acid, leukotriene B4) (see Lin, Q., et al., Biochemistry, 38(1): 185-90 (1999)

Perflourooctanoic acid

Fatty aryls (e.g., 4-iodophenylbutyrate, 4-chlorophenylbutyrate; clofibate; phenylbutyrate; naphthylacetate; 2,4-D; 4-chlorophenylacetate; phenylacetate; indoacetate)(see Pineau, T. et al., Biochem. Pharmacol., 53(4): 659-67 (1996);

Fibrates (e.g., beclobrate; bezafibrate; ciprofibrate; clofibrate; clofibride; etofibrate; fenofibrate; gemfibrozil; simfibrate) (see Staels, B. et al, Biochimie, 79(2-3): 95-9 (1997)).

U.S. Pat. No. 6,306,854 describes compounds that can serve as the PPARα agonists for use according to the present invention. The compounds have the general structure of formula XI, or a salt thereof, where the general structure is:

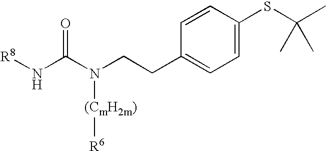

wherein m is from 0 to 20, R⁶ is selected from the group consisting of hydrogen and

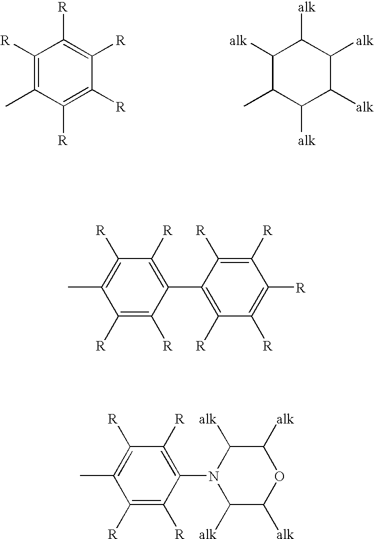

and R⁸ is selected from the group consisting of

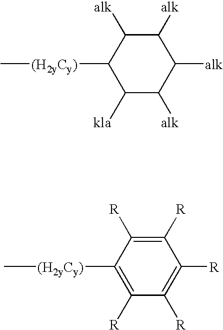

where y is 0, 1, or 2, each alk is independently hydrogen or alkyl group containing 1 to 6 carbon atoms, each R group is independently hydrogen, halogen, cyano, —NO₂, phenyl, straight or branched alkyl or fluoroalkyl containing 1 to 6 carbon atoms and. which can contain hetero atoms such as nitrogen, oxygen, or sulfur and which can contain functional groups such as ketone or ester, cycloalkyl containing 3 try 7 carbon atoms, or two R groups bonded to adjacent carbon atoms can, together with the carbon atoms to which they are bonded, form an aliphatic or aromatic ring or mufti ring system, and where each depicted ring has no more that 3 alk groups.

Examples of preferred compounds that have the structure of the above formula include:
2-(4-(2-(1-(4-biphenylethyl)-3-cyclohexylureido)ethyl)phenylthio)-2-methylpropionic acid,
2-(4-(2-(1-(2-(4-morpholinophenyl)ethyl-3-cyclohexylureido)ethyl)phenylthio)-2-methylpropionic acid;
2-(4-(2-(1-(cyclohexanebutyl)-3-cyclohexylureido)ethyl)phenylthio)-2-methylpropionic acid;
2-(4-(2-(1-heptyl-3-(2,4-difluorophenyl)ureido)ethyl)phenylthio)-2-methylpropionlc acid,
2-(4-(2-(1-(2-chloro-4-(2-trifluoromethylphenyl)phenylmethyl)-3-(cyclohexyl)ureido)ethyl)phenylthio)-2-methylpropionic acid, or a salt thereof.

In some embodiments, the PPARα agonist is selected from the following list:

WY-14,643, Fibrates (Most if not all are fibrates)
Clofibrate
Clofibride
Fenofibrate
clorofibrate
Benzafibrate
Ciprofibrate
Beclofibrate
(beclobrate)
etofibrate
simfibrate
gemfibrozil
Nafenopin
Benfluorex

pioglitazone

(−) DRF2725

BM-17.0744

docosahexanoic acid

JTT-501

Trichloroacetate

Dichloroacetate

DHEA

DHEA-S

leukotriene B4

Fatty acids and their derivatives which activate PPARα

Fatty acid ethanolamides and their derivatives which activate PPARα

ETYA

GW 9578

GW 7647

GW 2331

GW 9578

Tetradecylthioacetic acid (TTA)

8(S)-HETE
BRL 49653

Each of the above patents cited in this section are incorporated by reference herein with particular reference to the PPARα agonist compounds and compositions they disclose.

### Identification of PPARα Agonists

While many PPARα agonist compounds are known in the art, identification and characterization of suitable novel compounds that specifically or selectively bind PPARα can be accomplished by any means known in the art, such as, for example, electrophoretic mobility shift assays and competitive binding assays. Preferably PPARα specific binding compounds have at least 5-10 fold, preferably 10-100 fold, more preferably 100-500 fold, most preferably greater than 1000 fold specificity for PPARα compared to other PPAR subtypes. Mammalian PPAR subtypes (e.g., rat, mouse, hamster, rabbit, primate, guinea pig) are preferably used. More preferably, human PPAR subtypes are used.

Electrophoretic Mobility Shift Assays

Electrophoretic mobility shift assays can be used to determine whether test compounds bind to PPARα and affect its electrophoretic mobility. (Forman, et al., PNAS 94:4312 (1997) and Kliewer, et al., PNAS 91:7355 (1994)). Electrophoretic mobility shift assays involve incubating a PPAR-RXR with a test compound in the presence of a labeled nucleotide sequence. Labels are known to those of skill in the art and include, for example, isotopes such as, ³H, ¹⁴C, ³⁵S, and ³²P, and non-radioactive labels such as fluorescent labels or chemiluminescent labels. Fluorescent molecules which can be used to label nucleic acid molecules include, for example, fluorescein isothiocyanate and pentafluorophenyl esters. Fluorescent labels and chemical methods of DNA and RNA fluorescent labeling have been reviewed recently (Proudnikov et al., Nucleic Acids Res. 24:4535-42 (1996)).

Chemiluminescent labels and chemiluminescent methods of labeling DNA and RNA have been reviewed recently (Rihn et al., J. Biochem. Biophys. Methods 30:91-102 (1995)). Use of non-radioactive labeled probes directly for studying protein-polynucleotide interactions with EMSA has been described. (U.S. Pat. No. 5,900,358). The mixtures can be separated, run on a separate lane of a gel, and autoradiographed. For example, if a test compound does not result in a change in the bands seen in the control lane then the test compound is not a candidate PPARα specific binding compound. On the other hand, if a change in intensity in at least one of the bands is seen, then the compound is a candidate PPARα specific binding compound. (U.S. Pat. No. 6,265,160). The incubation mixture is then electrophoretically separated and the resulting gel exposed to X-ray film. The resulting autoradiograph may have one or more bands representing slowly migrating DNA-protein complexes. This control lane can indicate the mobility of the complex between the DNA probe and PPAR.

Monoclonal antibodies specific for PPAR subtypes can be used to identify PPARα specific binding compounds in modified electrophoretic mobility shift assays. Purified PPARβ, PPARα or PPARγ can be incubated with an appropriate amount of a test compound in the presence of RXR. For these assays, the test compound need not be labeled. PPAR subtype specific monoclonal antibodies can be incubated with the PPAR-RXR-test compound mixture. For instance, test compounds that bind PPAR induce supershifting of the PPAR-RXR complex on a gel (Forman, et al. PNAS 94:4312 (1997)) which can be detected by anti-PPAR monoclonal antibodies using a Western blot (immunoblot).

Generation of monoclonal antibodies has been previously described and can be accomplished by any means known in the art. (Buhring et al. in Hybridoma, Vol. 10, No. 1, pp. 77-78 (1991)). For example, an animal such as a guinea pig or rat, preferably a mouse is immunized with a purified PPAR subtype, the antibody-producing cells, preferably splenic lymphocytes, are collected and fused to a stable, immortalized cell line, preferably a myeloma cell line, to produce hybridoma cells which are then isolated and cloned. (U.S. Pat. No. 6,156,882).

Western blots generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind PPAR subtypes. These antibodies may be directly labeled or alternatively may be subsequently detected using labeled antibodies (e.g., labeled sheep anti-mouse antibodies) that specifically bind to the anti-PPAR antibodies.

The particular label or detectable group used in the assay is not a critical aspect of the invention, as long as it does not significantly interfere with the specific binding of the PPAR subtype specific ligand used in the assay. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵s, ¹⁴C, or ³²P), and calorimetric labels such as colloidal gold or colored glass or plastic beads (e.g., polystyrene, polypropylene, latex, etc.).

The molecules can be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead. In particular, one of ordinary skill in the art would appreciate that fluorescence resonance energy transfer (FRET) can be used.

Other assay formats include liposome immunoassays (LIA), which use liposomes designed to bind specific molecules (e.g., antibodies) and release encapsulated reagents or markers. The released chemicals can be then detected according to standard techniques (see Monroe et al., Amer. Clin. Prod. Rev. 5:34-41 (1986)).

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Competitive Binding Assays

In addition to electrophoretic mobility shift assays, competitive binding assays can be used to identify PPARα specific binding compounds. In competitive assays, the binding of test compounds to PPARα can be determined by measuring the amount of OEA that they displaced (competed away) from PPARα. Purified PPARβ, PPARα, and PPARγ receptors can be incubated with varying amounts of a test compound in the presence of labeled ligands specific for each PPAR subtype. For example, GW 2433 and L-783483 can be used in conjunction with PPARβ; GW 2331 or OEA can be used in conjunction with PPARα; and rosiglitazone, AD-5075, and SB-236636 can be used in conjunction with PPARγ. Specificity of the test compound for each PPAR subtype can be determined by detection of the amount of labeled ligand that remains bound to each PPAR after incubation with the test compound. Labels are discussed above.

High Throughput Screening of Candidate Compounds that Specifically Bind PPARα

In conjunction with the methods described above, identification of OEA-like compounds and OEA-like modulators can be accomplished via high throughput screening. Conventionally, new chemical entities with useful properties can be generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

High throughput screening methods involve providing a library containing a large number of potential PPARα specific binding compounds (candidate compounds). Such “combinatorial chemical libraries” can be then screened in one or more assays, as described herein, to identify those library members (particular chemical species or subclasses) that display a desired characteristic activity. The compounds thus identified can serve as conventional “lead compounds” or can themselves be used as potential or actual therapeutics.

a. Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library can be formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. 37(9):1233 (1994)).

Preparation and screening of combinatorial chemical libraries are well known to those of skill in the art. Such combinatorial chemical libraries include, but are not limited to, benzodiazepines (U.S. Pat. No. 5,288,514), diversomers such as hydantoins, benzodiazepines and dipeptides (Hobbs et al., PNAS. USA 90: 6909 (1993)), analogous organic syntheses of small compound libraries (Chen et al., J. Amer. Chem. Soc. 116: 2661 (1994)), oligocarbamates (Cho, et al., Science 261:1303 (1993)), and/or peptidyl phosphonates (Campbell et al., J. Org. Chem. 59: 658 (1994)), and small organic molecule libraries (see, e.g., benzodiazepines, Baum C&EN, January 18, page 33 (1993), thiazolidinones and metathiazanones U.S. Pat. No. 5,549,974, pyrrolidines U.S. Pat. Nos. 5,525,735 and 5,519,134, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devises are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Biosciences, Columbia, Md., etc.).

b. High Throughput Assays of Chemical Libraries

Many of the in vitro assays for compounds described herein are amenable to high throughput screening. Preferred assays thus detect activation of transcription (i.e., activation of mRNA production) by the test compound(s), activation of protein expression by the test compound(s), or binding to the gene product (e.g., expressed protein) by the test compound(s).

High throughput assays for the presence, absence, or quantification of particular protein products or binding assays are well known to those of skill in the art. Thus, for example, U.S. Pat. No. 5,559,410 discloses high throughput screening methods for proteins, and U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### Measuring Activation of PPARα

The ability of an OEA-like compound or OEA-like modulator to activate PPARα can be measured using any means known in the art. PPARα activators act by inducing PPARα-RXR heterodimer formation. The PPARα-RXR heterodimer then binds to DNA sequences containing AGGTCAnAGGTCA and activates PPAR target genes. Preferably PPARα activators activate PPARα by at least 5-10 fold, more preferably 10-100 fold, more preferably 100-500 fold, more preferably 500-100 fold, most preferably greater than 1000 fold above base level. PPARα can be transfected into cells. The transfected cells can be then exposed to candidate compounds. Any means known in the art can be used to determine whether PPARα is activated by the candidate compound, such as for example, by measuring levels of reporter gene expression and cell proliferation.

Transfection of PPAR into Cells

Any of the well-known procedures for introducing foreign nucleotide sequences into host cells may be used to transfect PPARα into cells such as, for example, calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). Methods of transfection have also been described in U.S. Pat. Nos. 5,616,745, 5,792,6512, 5,965,404, and 6,051,429 and in Current Protocols in Molecular Biology, Ausubel, et al., ed. (2001). It is only necessary that the particular genetic engineering procedure used be capable of successfully introducing at least one gene into the host cell capable of expressing PPARα. After the expression vector is introduced into the cells, the transfected cells can be cultured under conditions favoring expression of PPARα.

Detection of Reporter Gene Expression

Expression of reporter genes in response to compounds identified as binders of PPARα may also be used to measure PPARα activation. PPARα may be co-transfected with reporter genes known in the art such as, for example, luciferase, β-galactosidase, alkaline phosphatase, fluorescent green protein, or chloramphenicol acetyltransferase. The transfected cells can be exposed to appropriate concentrations of candidate compounds with OEA as a positive control. Reporter gene expression will be induced by compounds that bind and activate PPARα. Thus, compounds that induce reporter gene expression can be identified as activators of PPARα. (Forman, et al., PNAS 94:4312 (1997)). Preferably the compounds induce reporter gene expression at levels at least 5-10 fold, more preferably 10-100 fold, more preferably 100-500 fold, more preferably 500-1000 fold, most preferably greater than 1000 fold greater than the negative control.

Proliferation of PPARα Transfected Cells

PPARα activation may also be measured by proliferation of cells transfected with PPARα. Cell proliferation can be induced by compounds that bind and activate PPARα, such as, for example, OEA. Thus, PPARα transfected cells can be exposed to appropriate concentrations of candidate compounds with OEA as a positive control. Compounds that induce cells to proliferate can thereby be identified as activators of PPARα Cell proliferation can be measured, for example, by incorporation of 5′-bromo-2′deoxyuridine or 3H-thymidine as described in Jehl-Pietri, et al., Biochem J. 350:93 (2000) and Zoschke et al., Clin. Immunol. Immunopath. 32:29 (1984), respectively. Preferably the compounds induce cell proliferation at levels at least 5-10 fold, more preferably 10-100 fold, more preferably 100-500 fold, more preferably 500-1000 fold, most preferably greater than 1000 fold greater than the negative control.

Using analogous methods, one of ordinary skill in the art could similarly assess the activity of agents with respect to other PPAR receptors.

### CB1 Receptor Agonists for Use According to the Invention

A variety of CB1 receptor agonists are known to date; these include classical cannabinoids, such as, for example, Δ⁹-THC, non-classical cannabinoids, aminoalkylindoles and eicosanoids. The latter include the generally accepted endogenous CB1 receptor agonist anandamide.

CB1 Receptor Agonists for use according to the invention, include but are not limited to, compounds of Formula Ib as taught in U.S. Pat. No. 5,631,297.

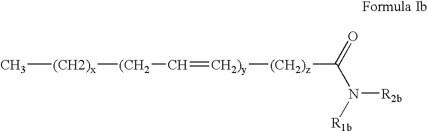

wherein R_{1b} and R_{2b} are each H or (CH₂)ₚ—(R_{4b}CH)_{q}—(CH₂)ᵣ—R_{3b}, wherein p, q and r are each an integer of from 0 to 10, preferably 1 to 4; and R_{3b} is OH, SH, CH₃, CH═CH₂, C≡CH, C≡N, F, Cl, Br or I, preferably OH, SH or F, more preferably OH; R_{4b} is H or (CH₂)ₛCH₃, wherein s is an integer from 0 to 10, preferably 0 to 4; provided that p+q+r+s is less than or equal to 10, preferably less than or equal to 4, preferably one of R₁ₐ and R_{2b} is H and the other of R₁ₐ and R_{2b} is (CH₂)ₚ(R_{4b}CH)_{q}(CH₂)ᵣR_{3b};

x is an integer of from 0 to 18, preferably 2 to 5;

y is an integer of from 0 to 8, preferably 2 to 4; and

z is an integer of from 0 to 18, preferably 2 to 5.

Non-limiting examples of the compounds represented by Formula (Ib) which can be employed in the present invention include the following:
arachidonylethanolamide
arachidonylethanethiolamide
arachidonylfluoroethylamide
7,10,13,16-docosatetraenylethanolamide
arachidonylpropanolamide
8, 11, 14-eicosatrienylethanolamide
4,7,10,13,16,19-Docosahexaenylethanolamide
arachidylfluoroethylamide
arachidonylamide
arachidonyl-1-methyl-ethanolamide
arachidonyl-2-methyl-ethanolamide,
gamma-linolenylethanolamide,
linoleylethanolamide

In accordance with this aspect of the present invention, there are disclosed pharmaceutical compositions and methods for treating pain comprising use of direct acting cannabinoid receptor agonists (e.g., arachidonylethanolamide (anandamide), (R)-(+)arachidonyl-1-hydroxy-2-propylamide, cis-7, 10,13,16-docosatetraenoylethanolamide, homo-delta-linoleyethanolamide, N-propyl-arachidonylethanolamide, N-ethyl-arachidonylethanolamide, and 2-arachidonylglycerol, and indirect acting FAAH inhibitors N-(4-hydroxyphenyl)-arachidonylamide, palmitylsulphonylfluoride, and arachidonyltrifluoromethylketone.

CB1 cannabinoid receptor agonists to be used according to the invention include those of the following formula:

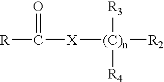

wherein X is N—R₁ or O; R is a saturated or unsaturated, chiral or achiral, cyclic or acyclic, substituted or unsubstituted hydrocarbyl group, wherein the hydrocarbyl group has 11 to 29 carbon atoms; R₁, R₃ and R₄ are selected independently from hydrogen, alkyl (C1-4), alkenyl (C2-4), alkynyl (C2-4), cycloalkyl (C3-6), or hydroxyalkyl group with from 2 to 4 carbon atoms; R₂ is OH or O—CO-alkyl, where the alkyl group has from 1 to 4 carbon atoms; and n is selected from 2 to 4.

### I. Cannabinoid Receptor Activity Screening.

While a great many CB1 agonist compounds are known in the art, additional suitable novel CB1 agonist compounds can be readily identified using methods known in the art. For instance, methods for screening compounds for CB1 agonist activity are well known to one of ordinary skill in the art. A variety of means may be used to screen cannabinoid CB1 receptor activity in order to identify the compounds for use according to the invention. A variety of such methods are taught in U.S. Pat. No. 5,747,524 and U.S. Pat. No. 6,017,919.

A. Ligand Binding Assays.

Ligand binding assays are well known to one of ordinary skill in the art. For instance, see, U.S. Patent Application No. US 2001/0053788 published on Dec. 20, 2001, U.S. Pat. No. 5,747,524, and U.S. Pat. No. 5,596,106 and (see, Felder, et al., Proc. Natl. Acad. Su., 90:7656-7660 (1993)) each of which is incorporated herein by reference. The affinity of an agent for cannabinoid CB1 receptors can be determined using membrane preparations of Chinese hamster ovary (CHO) cells in which the human cannabis CB1 receptor is stably transfected in conjunction with [³H]CP-55,940 as radioligand. After incubation of a freshly prepared cell membrane preparation with the [³H]-ligand, with or without addition of compounds of the invention, separation of bound and free ligand can be performed by filtration over glassfiber filters. Radioactivity on the filter was measured by liquid scintillation counting.

The cannabinoid CB1 agonistic activity of a candidate compound for use according to the invention can also be determined by functional studies using CHO cells in which human cannabinoid CB1 receptors are stably expressed. Adenylyl cyclase can be stimulated using forskolin and measured by quantifying the amount of accumulated cyclic AMP. Concomitant activation of CB1 receptors by CB1 receptor agonists (e.g., CP-55,940 or (R)-WIN-55,212-2) can attenuate the forskolin-induced accumulation of cAMP in a concentration-dependent manner. This CB1 receptor-mediated response can be antagonized by CB1 receptor antagonists. See, U.S. Patent Application No. US 2001/0053788 published on Dec. 20, 2001.

Samples rich in cannabinoid CB1 receptors and CB2 receptors, rat cerebellar membrane fraction and spleen cells can be respectively used (male SD rats, 7-9 weeks old). A sample (cerebellar membrane fraction: 50 μ.g/ml or spleen cells: 1(×10⁷ cells/ml), labeled ligand ([³H]Win55212-2, 2 nM) and unlabeled Win55212-2 or a test compound can be plated in round bottom 24 well plates, and incubated at 30° C. for 90 min in the case of cerebellar membrane fraction, and at 4° C. for 360 min in the case of spleen cells. As the assay buffer, 50 mM Tris solution containing 0.2% BSA can be used for cerebellar membrane fraction, and 50 mM Tris-HBSS containing 0.2% BSA can be used for spleen cells. After incubation, the samples are filtrated through a filter (Packard, Unifilter 24 GF/B) and dried. A scintillation solution (Packard, Microsint-20) can be added, and the radioactivity of the samples determined (Packard, Top count A9912V). The non-specific binding can be determined by adding an excess Win55212-2 (1 μM), and calculating specific binding by subtracting non-specific binding from the total binding obtained by adding the labeled ligand alone. The test compounds can be dissolved in DMSO to the final concentration of DMSO of 0.1%. EC₅₀ can be determined from the proportion of the specifically-bound test compounds, and the Kᵢ value of the test compounds can be calculated from EC₅₀ and K_{d} value of [3H]WIN55212-2. See, U.S. Pat. No. 6,017,919.

In one embodiment, the EC₅₀ for cannabinoid receptor binding is determined according to the method of Devane, et al., Science, 258: 1946-1949 (1992) and Devane, et al., J. Med. Chem., 35:2065 (1992). In this method, the a Devane, et al., Science, 258: 1946-1949 (1992) and Devane, et al., J. Med. Chem., 35:2065 (1992)bility of a compound to competitively inhibit the binding of a radiolabeled probe (e.g., ³H-HU-2430) is determined.

In other embodiments, the EC₅₀ of an agonist for the CB1 receptor is determined according to any one of the above ligand binding assay methods. In another embodiment, the EC₅₀ is according to any assay method which studies binding at physiological pH or physiologically relevant conditions. In another embodiment, the EC₅₀ is determined according to any assay method which studies binding at physiological pH and ionic strength. Preferred assay incubation temperatures range from 20° C.-37° C. Temperatures may be lower or higher. For instance, incubation temperatures of just a few degrees or 0° C. may be useful in preventing or slowing the degradation of enzymatically unstable ligands. Inhibitors of FAAH may also be added to protect antagonists from degradation.

B. Effect on N-Type Calcium Channel Currents.

Cannabinoid agonist activity can also be assessed by studying activation of the signal transduction pathway of the CB1 receptor, but in addition, effect other nerve cell organelles under control of the CB1 signaling pathway in vitro. Specifically, the agonists can close the N-type calcium channels (see, Mackie, K. and Hille, B., Proc. Natl. Acad. Sci., 89:3825-3829 (1992)). See, U.S. Pat. No. 5,596,106 which is incorporated herein by reference which teaches how to identify CB1 agonists on nerve cells by measuring current flow using a whole-cell voltage-clamp technique. A cannabinoid agonist (e.g., anandamide or WIN 55,212 will inhibit the N-type calcium channel via the CB1 receptor, thus decreasing the current to the voltage clamp of −65 pA. The addition of an CB1 receptor antagonist will oppose the action of the agonist.

C. Cannabinoid CB2 Receptor Binding Assay.

A variety of means may be used to screen cannabinoid CB2 receptor activity in order to identify compounds for use according to the invention. Methods of studying CB2 receptor binding are well known to one of ordinary skill in the art. For instance, binding to the human cannabinoid CB2 receptor can be assessed using the procedure of Showalter, et al., J. Pharmacol Exp Ther., 278(3):989-99 (1996)), with minor modifications as taught for instance in U.S. Patent Application No. 20020026050, published Feb. 28, 2002. Each of which is incorporated herein by reference.

In other embodiments, the EC₅₀ of an inventive compound for the CB2 receptor is determined according to any one of the above CB2 receptor ligand binding assay methods. In another embodiment, the EC₅₀ is according to any assay method which studies binding at physiological pH or physiologically relevant conditions. In another embodiment, the EC₅₀ is determined according to any assay method which studies binding at physiological pH and ionic strength. Preferred assay incubation temperatures range from 20° C.-37° C. Temperatures may be lower or higher. For instance, incubation temperatures of just a few degrees or 0° C. may be useful in preventing or slowing the degradation of enzymatically unstable ligands. Inhibitors of FAAH may also be added to protect antagonists from degradation.

### FAAH Inhibitors

Trifluoroketone inhibitors such as the compound of Formula IX are also contemplated for use in inhibiting FAAH to raise endogenous levels of OEA or treat the subject conditions and disorders.

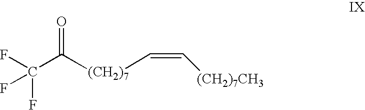

Such compounds are taught in U.S. Pat. No. 6,096,784 herein incorporated by reference.

Other compounds for use according to the invention include octylsulfonyl and octylphosphonyl compounds. See, Quistand, et al., in Toxicology and Applied Pharmacology, 179:57-63 (2002). See also Quistand, et al., in Toxicology and Applied Pharmacology, 173:48-55 (2001).

Other compounds for use according to the invention include the alpha-keto-oxazolpyridines which are reversible and extremely potent inhibitors of FAAH. See, Boger et al., PNAS USA, 97:5044-49 (2000). Suitable compounds include compounds of the Formula:

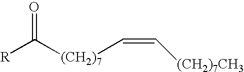

wherein R is an alpha-keto oxazolopyridinyl moiety such as

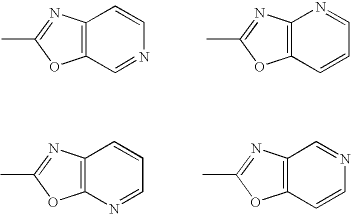

Boger et al. teach other suitable compounds for use according to the invention including substituted alpha-keto-heterocycle analogs of fatty acid amides. In particular, wherein R is an alpha-keto oxazolopyridinyl moiety and the fatty acid moiety is a homolog of oleic acid or arachidonic acid.

Other FAAH inhibitors for use according to the invention include fatty acid sulfonyl fluorides such as compound AM374 which irreversibly binds FAAH. See, Deutsch, et al., Biochem. Biophys Res Commun., 231:217-221 (1997).

Other preferred FAAH inhibitors include, but are not limited to, the carbamate FAAH inhibitors disclosed in Kathuria et al., Nat Med Jan; 9(1):76-81 (2003) incorporated herein by reference for the FAAH inhibitor compounds it discloses. Particularly preferred are selective FAAH inhibitors such as URB532 and URB597 disclosed therein.

FAAH inhibitors for use according to the invention include compounds of the following formula which inhibit FAAH:

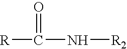

In the above formula, R is a polyunsaturated, substituted or unsubstituted hydrocarbyl group, wherein the hydrocarbyl group has from 18 to 22 carbon atoms; and R₂ is selected independently from substituted or unsubstituted cycloalkyl (C3-6) group and substituted or unsubstituted phenyl group. In some embodiments, the hydrocarbyl group R is a straight or branched chain C12-C26 fatty acid and may be saturated, monounsaturated, diunsaturated, or polyunsaturated.

In some embodiments, the fatty acid amide hydrolase inhibitor is selected from the group consisting of stearylsulfonyl fluoride, phenylmethylsulfonyl fluoride, trifluoromethyl ketones, diazomethylarachidonyl ketone, and pyrazinamide.

In some embodiments the FAAH inhibitor is represented by the following formula: A-B-C wherein: A is an α-keto heterocyclic pharmacophore for inhibiting the fatty acid amide hydrolase; B is a chain for linking A and C, said chain having a linear skeleton of between 3 and 9 atoms selected from the group consisting of carbon, oxygen, sulfur, and nitrogen, the linear skeleton having a first end and a second end, the first end being covalently bonded to the α-keto group of A, with the following proviso: if the first end of said chain is an α-carbon with respect to the α-keto group of A, then the α-carbon is optionally mono- or bis-functionalized with substituents selected from the group consisting of fluoro, chloro, hydroxyl, alkoxy, trifluoromethyl, and alkyl; and C is an activity enhancer for enhancing the inhibition activity of said α-keto heterocyclic pharmacophore, said activity enhancer having at least one π-unsaturation situated within a π-bond containing radical selected from a group consisting of aryl, alkenyl, alkynyl, and ring structures having at least one unsaturation, with or without one or more heteroatoms, said activity enhancer being covalently bonded to the second end of the linear skeleton of B, the π-unsaturation within the π-bond containing radical being separated from the α-keto group of A by a sequence of no less than 4 and no more than 9 atoms bonded sequentially to one another, inclusive of said linear skeleton (see, U.S. Patent Application Publication No. 20030092734, published on May 15, 2003, which is specifically incorporated herein by reference with respect to the FAAH inhibitors disclosed therein).

In other embodiments the FAAH inhibitor is an (oxime)carbamoyl fatty acid amide hydrolase inhibitor (see, U.S. Patent Application Publication No. 20030195226 which is specifically incorporated herein by reference and particularly with respect to the FAAH inhibitors disclosed therein). In particular embodiments, the FAAH inhibitor is selected from the group consisting of
pyridine-3-carbaldehyde, O-[[(4-undecyloxy-phenyl)amino]carbonyl]oxime;
pyridine-3-carbaldehyde, O-[[(4-nonyloxy-phenyl)amino]carbonyl]oxime;
4-fluorobenzaldehyde, O-[[(4-decyloxy-phenyl)amino]carbonyl]oxime;
4-fluorobenzaldehyde, O-[[(4-octyloxy-phenyl)amino]carbonyl]oxime;
benzaldehyde, O-[[(4-nonyloxy-phenyl)amino]carbonyl]oxime;
4-fluorobenzaldehyde, O-[[(4-nonyloxy-phenyl)amino]carbonyl]oxime;
3,4-difluorobenzaldehyde, O-[[(4-nonyloxy-phenyl)amino]carbonyl]oxime;
2,6-difluorobenzaldehyde, O-[[(4-nonyloxy-phenyl)amino]carbonyl]oxime;
2,4-difluorobenzaldehyde, O-[[(4-nonyloxy-phenyl)amino]carbonyl]oxime;
3-fluorobenzaldehyde, O-[[(4-nonyloxy-phenyl)amino]carbonyl]oxime;
pyridine-3-carbaldehyde, O-[[(4-nonyloxy-phenyl)amino]carbonyl]oxime;
benzaldehyde, O-[[(4-decyloxy-phenyl)amino]carbonyl]oxime;
pyridine-3-carbaldehyde, O-[[(4-decyloxy-phenyl)amino]carbonyl]oxime;
pyridine-3-carbaldehyde, O-[[(4-dodecyloxy-phenyl)amino]carbonyl]oxime;
benzaldehyde, O-[[(4-octyloxy-phenyl)amino]carbonyl]oxime;
2,3-difluorobenzaldehyde, O-[[(4-nonyloxy-phenyl)amino]carbonyl]oxime;
benzaldehyde, O-[[(4-undecyloxy-phenyl)amino]carbonyl]oxime;
2,4,5-trifluorobenzaldehyde, O-[[(4-nonyloxy phenyl)amino]carbonyl]oxime;
4-fluorobenzaldehyde, O-[[(4-phenoxyphenyl)amino]carbonyl]oxime;
benzaldehyde, O-[[(4-undecyloxy-phenyl)amino]carbonyl]oxime;
4-trifluoromethyl-benzaldehyde, O-[[(4-nonyloxy-phenyl)amino]carbon-yl]oxime;
benzaldehyde, O-[[(4-phenoxyphenyl)amino]carbonyl]oxime;
pyridine-3-carbaldehyde, O-[[(4-heptyloxy-phenyl)amino]carbonyl]oxime;
benzaldehyde, O-[[[4-(2-phenylethoxy)phenyl]amino]carbonyl]oxime;
2-fluoro-3-trifluoromethyl-benzaldehyde, O-[[(4-nonyloxy-phenyl)ami-no]carbonyl]oxime;
(4-undecyloxy-phenyl)-carbamic acid phenyl ester;
propan-2-one, O-[[(4-heptyloxy-phenyl)amino]carbonyl]oxime;
propan-2-one, O-[[(4-nonyloxy-phenyl)amino]carbonyl]oxime;
benzaldehyde, O-[[[4-(phenylmethoxy)phenyl]amino]carbonyl]oxime;
4-fluorobenzaldehyde, O-[[[4-(2-phenylethoxy)phenyl]amino]carbonyl]-oxime;
2-fluoro-5-trifluoromethyl-benzaldehyde, O-[[(4-nonyloxy-phenyl)amino]carbonyl]oxime;
4-fluorobenzaldehyde, O-[[[4-(phenylmethoxy)phenyl]amino]carbonyl]oxime;
3-pyridinecarboxaldehyde, O-[[(3-phenoxyphenyl)amino]carbonyl]oxime-;
4-fluorobenzaldehyde, O-[[[4-(3-phenylpropoxy)phenyl]amino]carbonyl-]oxime;
benzaldehyde, O-[[(3-phenoxyphenyl)amino]carbonyl]oxime;
4-fluorobenzaldehyde, O-[[(4-pentyloxy-phenyl)amino]carbonyl]oxime;
4-fluorobenzaldehyde, O-[[(4-butoxy-phenyl)amino]carbonyl]oxime;
pyridine-3-carbaldehyde, O-[[(4-heptyloxy phenyl)amino]carbonyl]oxime;
3-pyridinecarboxaldehyde, O-[[(4-phenoxyphenyl)amino]carbonyl]oxime-;
benzaldehyde, O-[[[4-(3-phenylpropoxy)phenyl]amino]carbonyl]oxime;
4-fluorobenzaldehyde, O-[[(4-pentyloxy-phenyl)amino]carbonyl]oxime;
4-fluorobenzaldehyde, O-[[(4-dodecyloxy-phenyl)amino]carbonyl]oxime-;
propan-2-one, O-[[(4-decyloxy-phenyl)amino]carbonyl]oxime;
benzaldehyde, O-[[(4-dodecyloxy-phenyl)amino]carbonyl]oxime;
benzaldehyde, O-[[(4-pentyloxy-phenyl)amino]carbonyl]oxime;
2,4-difluorobenzaldehyde, benzaldehyde, O-[[(4-nonanoylamino-phenyl-amino]carbonyl]oxime;
4-fluorobenzaldehyde, O-[[(4-heptyloxy-phenyl)amino]carbonyl]oxime;
benzaldehyde, O-[[(4-pentyloxy-phenyl)amino]carbonyl]oxime;
propan-2-one, O-[[(4-undecyloxy-phenyl)amino]carbonyl]oxime;
propan-2-one, O-[[(4-dodecyloxy-phenyl)amino]carbonyl]oxime;
pyridine-3-carbaldehyde, O-[[(4-pentyloxy-phenyl)amino]carbonyl]oxime;
benzaldehyde, O-[[(4-propoxy-phenyl)amino]carbonyl]oxime;
benzaldehyde, O-[[(4-heptyloxy-phenyl)amino]carbonyl]oxime;
benzaldehyde, O-[[(4-butoxy-phenyl)amino]carbonyl]oxime;
benzaldehyde, O-[[(4-hexyloxy-phenyl)amino]carbonyl]oxime;
propan-2-one, O-[[(4-heptyloxy-phenyl)amino]carbonyl]oxime;
pyridine-3-carbaldehyde, O-[[(4-hexyloxy-phenyl)amino]carbonyl]oxime; and
pyridine-3-carbaldehyde, O-[[(4-butoxy-phenyl)amino]carbonyl]oxime.

Other FAAH inhibitors for use according to the invention are characterized by a carbamic template substituted with alkyl or aryl groups at their O- and N-termini. Most such compounds inhibit FAAH, but not several other serine hydrolases, with potencies that depend on the size and shape of the substituents. Preferred compounds have a lipophilic N-alkyl substituent (such as n-butyl or cyclohexyl) and a bent O-aryl substituent. N-cyclohexylcarbamic acid biphenyl-3-yl ester is an exemplary such compound. See Tarzia et al., J Med. Chem. 46(12):2352 (2003). Method of screening compounds for FAAH inhibitory activity are well known in the art.

In another embodiment, the FAAH inhibitor is a bisarylimidazolyl fatty acid amide hydrolase inhibitor as disclosed in U.S. Patent Application Publication No. 20020188009, published Dec. 12, 2002, which is specifically incorporated herein by reference and particularly with respect to the FAAH inhibitors disclosed therein). In some embodiments, the FAAH inhibitor is selected from the group consisting of [6-(2-Methyl-4,5-diphenyl-imidazol-1-yl)-hexyl]-carbamic acid 2-fluoro-phenyl ester; [6-(2-Ethyl-4,5-diphenyl-imidazol-1-yl)-hexyl]-car-bamic acid tert-butyl ester; 6-(2-Ethyl-4,5-diphenyl-imidazol-1-yl)-hexyl]-1-carbamic acid sec-butyl ester; [6-(2-Methyl-4,5-diphenyl-imidazol-1-yl)-h-exyl]-carbamic acid benzyl ester; 2-Propanone,O—[6-(2-methyl-4,5-diphenyl-1H-imidazol-1-yl)hexyl]amino]carbonyl]oxime; [6-(2-Methyl-4,5-diphenyl-imi-dazol-1-yl)-hexyl]-carbamic acid methyl ester; 6-(2-Methyl-4,5-diphenyl-im-idazol-1-yl)-hexyl]-carbamic acid phenyl ester; [6-(2-Methyl-4,5-diphenyl-imidazol-1-yl)-hexyl]-carbamic acid 4-fluoro-phenyl ester; [6-(2-Methyl-4,5-diphenyl-imidazol-1-yl)-hexyl]-carbamic acid 2,4-difluoro-phenyl ester; [6-(2-Methyl-4,5-diphenyl-imidazol-1-yl)-hexyl-]-carbamic acid 4-chloro-phenyl ester; [6-(2-Methyl-4,5-diphenyl-imidazol-1-yl)-hexyl]-carbamic acid 4-methoxy-phenyl ester; [6-(2-Methyl-4,5-diphen-yl-imidazol-1-yl)-hexyl]-carbamic acid o-tolyl ester; [6-(2-Methyl-4,5-diphenyl-imidazol-1-yl)-hexyl]-carbamic acid 4-cyano-phenyl ester; [6-(2-Methyl-4,5-diphenyl-imidazol-1-yl)-hexyl]-carbamic acid 2,6-dimethoxy-phenyl ester; [6-(2-Methyl-4,5-diphenyl-imidazol-1-yl)-hexyl]-carbamic acid 2-methoxy-phenyl ester; [7-(2-Methyl-4,5-diphen-yl-imidazol-1-yl)-heptyl]-carbamic acid methyl ester; [7-(2-Methyl-4,5-diphenyl-imidazol-1-yl)-heptyl]-carbamic acid ethyl ester; [7-(2-Methyl-4,5-diphenyl-imidazol-1-yl)-heptyl]-carbamic acid phenyl ester; [7-(2-Methyl-4,5-diphenyl-imidazol-1-yl)-heptyl]-carbamic acid 4-fluoro-phenyl ester; [7-(2-Methyl-4,5-diphenyl-imidazol-1-yl)-hept-yl]-carbamic acid 2-fluoro-phenyl ester; [7-(2-Methyl-4,5-diphenyl-imidazo-l-1-yl)-heptyl]-carbamic acid 2,4-difluoro-phenyl ester; [7-(2-Methyl-4,5-diphenyl-imidazol-1-yl)-heptyl]-carbamic acid 4-chloro-phenyl ester; [7-(2-Methyl-4,5-diphenyl-imidazol-1-yl)-heptyl]-c-arbamic acid 4-methoxy-phenyl ester; [7-(2-Methyl-4,5-diphenyl-imidazol-1-yl)-heptyl]-carbamic acid o-tolyl ester; [7-(2-Methyl-4,5-diphenyl-imidazo-l-1-yl)-heptyl]-carbamic acid 4-cyano-phenyl ester; [7-(2-Methyl-4,5-diphenyl-imidazol-1-yl)-heptyl]-carbamic acid 2,6-dimethoxy-phenyl ester; [7-(2-Methyl-4,5-diphenyl-imidazol-1-yl)-hept-yl]-carbamic acid 2-methoxy-phenyl ester; [5-(2-Methyl-4,5-diphenyl-imidaz-ol-1-yl)-pentyl]-carbamic acid ethyl ester; [5-(2-Methyl-4,5-diphenyl-imid-azol-1-yl)-pentyl]-carbamic acid phenyl ester; [5-(2-Methyl-4,5-diphenyl-1-midazol-1-yl)-pentyl]-carbamic acid 4-fluoro-phenyl ester; [5-(2-Methyl-4,5-diphenyl-imidazol-1-yl)-pentyl]-carbamic acid 2,4-difluoro-phenyl ester; [5-(2-Methyl-4,5-diphenyl-imidazol-1-yl)-penty-l]-carbamic acid 2-fluoro-phenyl ester; [5-(2-Methyl-4,5-diphenyl-imidazol-l-1-yl)-pentyl]-carbamic acid 4-chloro-phenyl ester; [5-(2-Methyl-4,5-diphenyl-imidazol-1-yl)-pentyl]-carbamic acid 4-methoxy-phenyl ester; [5-(2-Methyl-4,5-diphenyl-imidazol-1-yl)-pentyl]-carbamic acid o-tolyl ester; [5-(2-Methyl-4,5-diphenyl-imidazol-1-yl)-pent-yl]-carbamic acid 4-cyano-phenyl ester; [5-(2-Methyl-4,5-diphenyl-imidazol-l-1-yl)-pentyl]-carbamic acid 2,6-dimethoxy-phenyl ester; [5-(2-Methyl-4,5-diphenyl-imidazol-1-yl)-pentyl]-carbamic acid 2-methoxy-phenyl ester; [6-(2-Methyl-4,5-diphenyl-imidazol-1-yl)-hexyl]-c-arbamic acid 3,4-difluoro-phenyl ester; {6-[4,5-Bis-(4-fluoro-phenyl)-2-me-thyl-imidazol-1-yl]-hexyl}-carbamic acid 2-fluoro-phenyl ester; {6-[4,5-Bis-(4-fluoro-phenyl)-2-methyl-imidazol-1-yl]-hexyl}-carbamic acid 2,6-difluoro-phenyl ester; [6-(2-Methyl-4,5-diphenyl-imidazol-1-yl)-hexyl]-carbamic acid ethyl ester; Benzaldehyde, O—[6-(2-methyl-4,5-dipheny-1-1H-imidazol-1-yl)hexyl]amino]carbonyl]oxime; 4-Fluorobenzaldehyde,O—[6-(−2-methyl-4,5-diphenyl-1H-imidazol-1-yl)hexyl]amino]carbonyl]oxime; 2-Nitrobenzaldehye, O—[6-(2-methyl-4,5-diphenyl-1H-imidazol-1-yl)hexyl]am-ino]carbonyl]oxime; 3-Nitrobenzaldehyde, O—[6-(2-methyl-4,5-diphenyl-1H-im-idazol-1-yl)hexyl]amino]carbonyl]oxime; 4-Nitrobenzaldehyde, O—[6-(2-methyl-4,5-diphenyl-1H-imidazol-1-yl)hexyl]amino]carbonyl]oxime; 3-Pyridinecarboxaldehyde, O—[6-(2-methyl-4,5-diphenyl-1H-imidazol-1-yl)he-xyl]amino]carbonyl]oxime; {4-[2-(2-Methyl-4,5-diphenyl-imidazol-1-yl)-etho-xy]-phenyl}-carbamic acid 3,4-difluoro-phenyl ester; {4-[2-(2-Methyl-4,5-diphenyl-imidazol-1-yl)-ethoxy]-phenyl}-carbamic acid 4-chloro-phenyl ester; {4-[3-(2-Methyl-4,5-diphenyl-imidazol-1-yl)-propox-y]-phenyl}-carbamic acid 3,4-difluoro-phenyl ester; {4-[3-(2-Methyl-4,5-diphenyl-imidazol-1-yl)-propoxy]-phenyl}-carbamic acid 4-methoxy-phenyl ester; {4-[3-(2-Methyl-4,5-diphenyl-imidazol-1-yl)-propoxy]-phenyl}-carbamic acid 4-chloro-phenyl ester; {4-[3-(2-Methyl-4,5-diphenyl-imidazol-1-yl)-propoxy]-phenyl}-carbamic acid 2-methoxy-phenyl ester; {4-[3-(2-Methyl-4,5-diphenyl-imidazol-1-yl)-propoxy]-phenyl}-carbamic acid 3-chloro-phenyl ester; {4-[2-(2-Methyl-4,5-diphenyl-imidazol-1-yl)-ethoxy]-phenyl}-carbamic acid phenyl ester; {4-[2-(2-Methyl-4,5-diphenyl-imidazol-1-yl)-ethoxy]-phenyl}-carbamic acid 2-fluoro-phenyl ester; {4-[2-(2-Methyl-4,5-diphenyl-imidazo-l-1-yl)-ethoxy]-phenyl}-carbamic acid 4-fluoro-phenyl ester; {4-[3-(2-Methyl-4,5-diphenyl-imidazol-1-yl)-propoxy]-phenyl}-carbamic acid phenyl ester; {4-[2-(2-Methyl-4,5-diphenyl-imidazol-1-yl)-ethoxy]-ph-enyl}-carbamic acid 4-methoxy-phenyl ester; {4-[3-(2-Methyl-4,5-diphenyl-1-midazol-1-yl)-propoxy]-phenyl}-carbamic acid 2-fluoro-phenyl ester; {4-[3-(2-Methyl-4,5-diphenyl-imidazol-1-yl)-propoxy]-phenyl}-carbamic acid 2,6-difluoro-phenyl ester; {4-[2-(2-Methyl-4,5-diphenyl-imidazol-1-y-l)-ethoxy]-phenyl}-carbamic acid ethyl ester; [1-Methyl-6-(2-methyl-4,5-di-phenyl-imidazol-1-yl)-hexyl]-carbamic acid 2-fluoro-phenyl ester; [1-Ethyl-6-(2-methyl-4,5-diphenyl-imidazol-1-yl)-hexyl]-carbamic acid 2-fluoro-phenyl ester; [1-Isopropyl-6-(2-methyl-4,5-diphenyl-imidazol-1-y-l)-hexyl]-carbamic acid 2-fluoro-phenyl ester and [6-(2-Methyl-4,5-diphenyl-1-imidazol-1-yl)-1-phenyl-hexyl]-carbamic acid 2-fluoro-phenyl ester.

In another embodiment, the FAAH inhibitor is a haloenol lactone compound of the following formula:

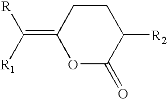

wherein R is hydrogen, R₁ is a halogen and R₂ is selected from the group consisting of aryl, aryloxy, and heteroaryl radicals. In one such embodiment, the haloenol lactone is E-6-(bromomethylene) tetrahydro-3-(1-naphthalenyl)-2H-pyrane-2-one. See U.S. Pat. No. 6,525,090 which is incorporated by reference in its entirety particularly with respect to the disclosure of such compounds.)

### Methods of Screening Compounds for FAAH Inhibitory Activity.

While many FAAH inhibitory compounds are known in the art, additional suitable FAAH inhibitory compounds can be readily identified using methods known in the art. Methods for screening compounds for FAAH inhibitory activity in vitro are well known to one of ordinary skill in the art. Such methods are taught in Quistand, et al., in Toxicology and Applied Pharmacology, 179:57-63 (2002); Quistand, et al., in Toxicology and Applied Pharmacology, 173: 48-55 (2001); and Boger, et al., PNAS USA, 97:5044-49 (2000).

Methods for screening compounds for FAAH inhibitory activity in vivo and increased endogenous cannabinoid levels or activity are known to one of ordinary skill in the art. Such methods include measurement of fatty acid ethanolamides in tissue and are taught in Quistand, et al., in Toxicology and Applied Pharmacology, 179: 57-63 (2002); Quistand, et al., in Toxicology and Applied Pharmacology, 173: 48-55 (2001); Boger, et al., PNAS USA, 97:5044-49 (2000). See, U.S. Pat. No. 6,096,784. See also PCT Publication WO 98/24396. See, Cravatt, et al., PNAS, 98:9371-9376 (2001).

### Anandamide Transport Inhibitors

The anandamide transport inhibitors for use according to the invention include amide and ester analogs of anandamide and exhibit the tail, central and head pharmacophore portions represented by Structural Formula: X—Y-Z wherein the tail portion X is a fatty acid chain remnant, central portion Y is an amide or ester radical and head portion Z is selected form the group consisting of hydrogen, alkyl, hydroxy alkyl, aryl, hydroxy aryl, heterocyclic and hydroxy heterocyclic radicals. See U.S. Patent Application Publication No. 20030149082 published on Aug. 7, 2003 (application Ser. No. 328,742). U.S. Patent Application Publication No. 20030149082 is herein incorporated by reference in its entirety and in particular with respect to the anandamide transport inhibitors and anandamide transport inhibition assays disclosed therein.

Assays for anandamine transport inhibition are well known to one of ordinary skill in the art. Exemplary methods for screening such compounds and identifying novel suitable compounds with such inhibitory activity are taught in U.S. Patent Application Publication No. 20040048907 published on Mar. 11, 2004 (U.S. patent Ser. No. 439,347, filed May 15, 2003), PCT Patent Publication No. WO 03/097573, and U.S. Patent Application Publication No. 20030149082. Such assays can be used to identify other anandamide transport inhibitors for use according to the present invention. Exemplary anandamide transport inhibitors for use according to the invention include M404, AM1172, OMDM1 and UCM707. U.S. Patent Application Publication No. 20040048907 and PCT Patent Publication No. WO 03/097573 are herein incorporated by reference in their entirety and in particular with respect to the anandamide transport inhibitors and anandamide transport inhibition assays disclosed therein.

### Methods for Assessing Pain in Evaluating Therapeutic Dosages and Identifying Further Suitable PPARα Agonists

The diagnosis and assessment of neuropathic pain is well known to one of ordinary skill in the art. Pain can be identified and assessed according to its onset and duration, location and distribution, quality and intensity, and secondary signs and symptoms (e.g., mood, emotional distress, physical or social functioning), and triggering stimulus or lack thereof. For human subject, often subjective pain assessment scales are used to measure intensity. Such scales may grade pain intensity verbally ranging from no pain-mild pain-moderate pain-severe pain-very severe pain and worst possible pain, or on a numeric scale from 1 (no pain) to 5 (moderate pain) to 10 (worst possible pain).

Suitable animal models for testing the ability of agents to treat neuropathic pain are also known to one of ordinary skill in the art. Such methods have been the subject of recent review (Wang et al. Advanced Drug Delivery Reviews 55:949 (2003)) which is incorporated by reference herein in its entirety. Methods of assessing neuropathic pain include 1) the weight drop or contusion model of Allen; 2) the photochemical SCI model: 3) the excitotoxic spinal cord injury model; 4) the neuroma model; 5) the chronic constriction injury model of Bennett; 6) the partial sciatic nerve ligation model; 7) the L5/L6 spinal ligation model; 8) the sciatic cryoneurolysis model; and 9) the sciatic inflammatory neuritis model. In addition there are a variety of models for studying the neuropathic pain of diabetes polyneuropathy; toxic neuropathies; and various bone cancer models.

### Pharmaceutical Compositions.

Another aspect of the present invention provides pharmaceutical compositions which comprises a PPARα agonist and a CB2 or CB1 cannabinoid receptor agonist (e.g., anandamide) or a PPARα agonist and a FAAH inhibitor or a PPARα agonist and an anandamide transport inhibitor. In some embodiments, the PPARα agonist is a selective PPARα agonist, an OEA-like compound or an OEA-like modulator. The composition can further comprise a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend in part on the nature and severity of the conditions being treated and on the nature of the active ingredient. An exemplary route of administration is the oral route. The compositions may be conveniently presented in unit dosage form and prepared by any of the methods well-known in the art of pharmacy.

In practical use, the active agents for use according to the invention (e.g., CB1 or CB2 receptor agonists, PPARα agonists, FAAH inhibitors, anandamide transport inhibitors) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers can be employed. If desired, tablets may be coated by standard aqueous or nonaqueous techniques. Such compositions and preparations can contain at least 0.1 percent of active compounds. The percentage of active compound in these compositions may, of course, be varied and may conveniently be between about 2 percent to about 60 percent of the weight of the unit. The amount of active compound in such therapeutically useful compositions is such that a therapeutically effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. To prevent breakdown during transit through the upper portion of the GI tract, the composition may be an enteric coated formulation.

### Administration

The pharmaceutical compositions of the invention may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The active agents are administered in therapeutically effective amounts. The active agents can each be effective over a wide dosage range. For example, in the treatment of adult humans, dosages from about 10 to about 1000 mg, about 100 to about 500 mg or about 1 to about 100 mg of any particular agent may be needed. Doses of the 0.05 to about 100 mg, and more preferably from about 0.1 to about 100 mg, per day may be used. A most preferable dosage is about 0.1 mg to about 70 mg per day. In choosing a regimen for patients, it may frequently be necessary to begin with a dosage of from about 2 to about 70 mg per day and when the condition is under control to reduce the dosage as low as from about 0.1 to about 10 mg per day. For example, in the treatment of adult humans, dosages from about 0.05 to about 100 mg, preferably from about 0.1 to about 100 mg, per day may be used. The exact dosage will depend upon the agent, mode of administration, on the therapy desired, form in which administered, the severity and condition of the subject to be treated and the body weight of the subject to be treated, and the preference and experience of the physician or veterinarian in charge.

Generally, active agents can be dispensed in unit dosage form comprising preferably from about 0.1 to about 100 mg of active ingredient together with a pharmaceutically acceptable carrier per unit dosage. Usually, dosage forms suitable for oral, nasal, pulmonary or transdermal administration comprise from about 0.001 mg to about 1000 mg, preferably from about 0.1 mg to about 100 mg of the compounds admixed with a pharmaceutically acceptable carrier or diluent. For storage and use, these preparations preferably contain a preservative to prevent the growth of microorganisms.

Administration of an appropriate amount of the compounds may be by any means known in the art such as, for example, oral or rectal, parenteral, intraperitoneal, intravenous, subcutaneous, subdermal, intranasal, or intramuscular. In some embodiments, administration is transdermal. In yet other embodiments, administration is topical. An appropriate amount or dose of the candidate compound may be determined empirically as is known in the art. For example, with respect to neuropathic pain, a therapeutically effective amount is an amount sufficient to reduce the severity of pain as measured by subjective or objective indicia in the subject over time. The candidate compound can be administered as often as required to reduce or control pain, for example, hourly, every two, three, four, six, eight, twelve, or eighteen hours, daily in the case of chronic pain, or according to the actual or subjective perception of pain so as to reduce it to a more tolerable level, or in advance of activities likely to exacerbate the pain.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Injection solutions and suspensions can be prepared from sterile powders, granules, and tablets of the kind previously described. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

With respect to transdermal routes of administration, methods for transdermal administration of drugs are disclosed in Remington's Pharmaceutical Sciences, 17th Edition, (Gennaro et al. Eds., Mack Publishing Co., 1985). Dermal or skin patches are a preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012, 6,261,595, and 6,261,595. Each of which is incorporated by reference in its entirety.

Preferred patches include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

The active agents of the present invention can be useful in the treatment, prevention, suppression of pain and may be used in combination with other compounds or with other drugs that are useful in the treatment, prevention, suppression or relief of pain or inflammation. Such other drugs (e.g., NSAIDs such as aspirin, aceteominophen, diclofenac, indomethacin, piroxicam, and nabumetone, ketoprofen, naproxen, ibuprofen; COX-2 inhibitors such as celecoxib, and opiates such as morphine, codeine, hydromorphone, oxycodone, oxymorphone, hydrocodone, meperidine, fentanyl, and methadone) may be administered, by a route and in an amount commonly used therefore, contemporaneously or sequentially with a compound of the invention or co-formulated therewith. When the active agent is used contemporaneously with one or more other drugs, a pharmaceutical composition in unit dosage form containing such other drugs and the compound is preferred. When used in combination with one or more other active ingredients, the compound of the present invention and the other active ingredients may be used in lower doses than when each is used singly. Accordingly, the pharmaceutical compositions of the present invention include those that contain one or more other active ingredients, in addition to the compounds disclosed above.

The pharmaceutically or physiologically acceptable salts include, but are not limited to, a metal salts such as sodium salt, potassium salt, lithium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

The following examples are provided to illustrate, and not to limit, the invention.

### EXAMPLES

### Example 1

### Role of PPARα in Modulating Pain

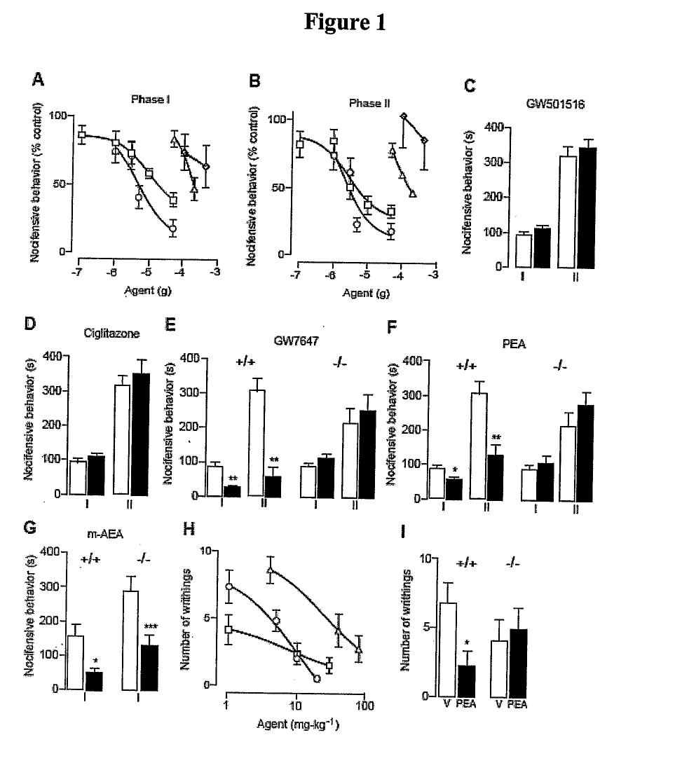

Administration of formalin into the mouse hind paw evokes a pain behavior consisting of two temporally distinct phases of licking and flexing of the injected limb (Dubuisson, et al., Pain 4, 161-74 (1977)). The first phase, which starts immediately after formalin injection and lasts 5-10 min, is due to activation of nociceptive fibers and is accompanied by the local release of nitric oxide and adenosine [Dickenson, et al., Neurosci Lett 83, 207-11 (1987); Omote, et al., Brain Res 787, 161-4 (1998); Omote et al., 2000; Liu, et al., J Neurochem 80, 562-70 (2002)]. Following a quiescent interval of 10-15 minutes, a second phase of nociceptive behavior appears, which is associated with local inflammation [Rosland, et al., Pain 42, 235-42 (1990)] and central sensitization [Coderre, et al., J Neurosci 12, 3665-70 (1992)]. PEA attenuates both phases in a dose-dependent manner [Calignano, et al., Nature 394, 277-81 (1998); Calignano, et al., Prog Brain Res 129, 471-82 (2000); Jaggar, et al., Pain 76, 189-99 (1998)]. This antinociceptive effect precedes PEA-induced anti-inflammation, which requires at least 2 hours for full expression [Mazzari, et al., Eur J Pharmacol 300, 227-36 (1996)], and is blocked by the cannabinoid CB₂ antagonist SR144528 [Calignano, et al., Nature 394, 277-81 (1998); Calignano, et al, Eur J Pharmacol 419, 191-8. (2001); Farquuar-Smith, 2002]. However, PEA does not bind to CB₂ receptors [Showalter, et al., J Pharmacol Exp Ther 278, 989-99 (1996); Calignano, et al., Nature 394, 277-81 (1998)] and the mechanism underlying its antinociceptive properties remains unknown. The effects of PEA in C57BL/6J mice in which the PPARα gene had been deleted by homologous recombination (PPARα^{-/-})[Lee, et al., Mol Cell Biol 15, 3012-22 (1995)] were examined to determine whether PPARα contributes to these properties. Intraplantar (i.pl.) formalin injection produced equivalent nocifensive responses in wild-type and PPARα^{-/-} mice (FIG. 1f), but PEA treatment (50 μg, i.pl.) attenuated these responses only in wild-type animals (FIG. 1f).

Although PPARα is known to regulate the inflammatory response [Devchand, et al., Nature 384, 39-43. (1996); Cabrero, et al., Curr Drug Targets Inflamm Allergy 1, 243-8 (2002); Berger, et al., Annu Rev Med 53, 409-35 (2002); Chinetti, et al., Inflamm Res 49, 497-505 (2000)], its roles in the modulation of acute nociceptive pain remain unexplored. A series of structurally diverse PPAR-α agonists were tested for their ability to reduce pain behavior in an animal model of chemical tissue damage. Injection of formalin into the mouse hind paw evokes a nocifensive behavior consisting of two distinct phases. The first phase results from direct activation of nociceptive fibers and starts immediately after formalin administration, while the second phase develops over a period of approximately 60 min and is associated with local inflammation and central sensitization. Two high-affinity PPAR-α agonists, GW7647 (0.1-50 μg, intraplantar, i.pl.) and Wy-14643 (50-200 μg, i.pl.) (Willson, et al., J Med Chem 43, 527-50 (2000)) attenuated first- and second-phase nociception in a dose-dependent manner when injected into the paw together with formalin (FIG. 1A,B). In ten experiments, average half-maximal effective doses (ED₅₀) to reduce first-phase pain were 9.9 μg for GW7647 and 97.0 μg for Wy-14643. In contrast, the weak PPAR-α agonist fenofibric acid (Willson, et al., J Med Chem 43, 527-50 (2000)) was ineffective at all doses tested (100-400 μg, i.pl.) (FIG. 1A,B). Selective agonists of other PPAR isoforms—PPAR-δ (GW501516, 50 μg, i.pl.) and PPAR-γ (ciglitazone, 50 μg, i.pl.)—were also inactive (FIG. 1C,D). Showing the role of PPAR-α in these effects, mutant mice lacking this receptor (PPAR-α^{-/-} mice) (Lee, et al., Mol Cell Biol 15, 3012-22 (1995))failed to respond to GW7647 (50 μg, i.pl.) (FIG. 1E). However, PPAR-α^{-/-} mice exhibited a normal response to formalin (FIG. 1E,F), which was reversed by the cannabinoid analgesic (R)-methanandamide (Calignano, et al., Nature 394, 277-81 (1998)) (m-AEA, 50 μg, i.pl.) (FIG. 1G).

The natural fatty-acid amide, palmitoylethanolamide (PEA), exerts potent antiinflammatory, antinociceptive and antihyperalgesic effects in rodents (Calignano, et al., Nature 394, 277-81 (1998); Mazzari, et al., Eur J Pharmacol 300, 227-36 (1996); Calignano, et al, Eur J Pharmacol 419, 191-8. (2001); Jaggar, et al., Pain 76, 189-99 (1998). Here, we show PEA activates PPARα in vitro and inhibits inflammatory responses by engaging this receptor. In agreement with this idea, we found that the antinociceptive effect of PEA (ED₅₀=7.4 μg, i.pl.) in the formalin model (FIG. 1A,B) was absent in PPARα^{-/-} mice (FIG. 1F).

We have also found (data set forth in the corresponding priority provisional application) that there is an absence of anti-inflammatory effects when vehicle (saline/PEG/Tween 80 (V; 90/5/5)), PEA (PEA; 50 μg/paw), or GW7647 (GW; 50 μg/paw) at 45 minutes after intraplantar administration of formalin (5% v/v, 10 μl) compared to untreated control (UN) C57BL/6J mice **, P<0.01, ANOVA followed by Dunnett's test (n=5).

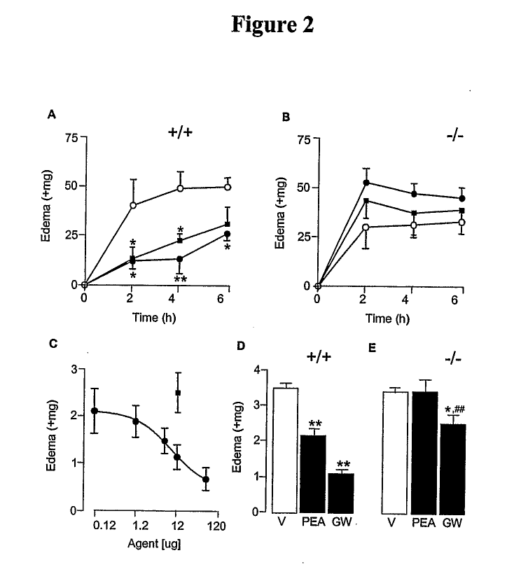

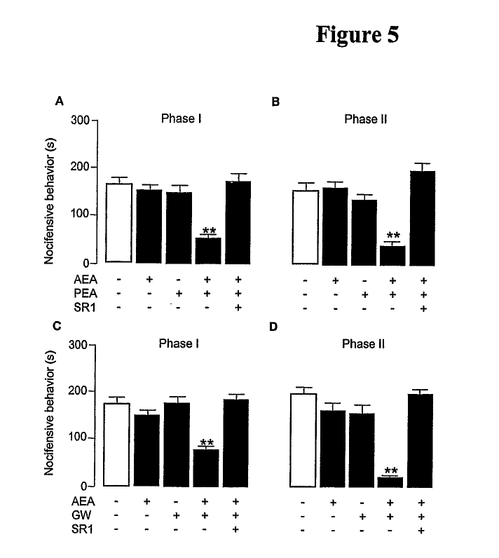

In addition to rapid-onset analgesia, PEA also exerts prolonged anti-inflammatory effects [Benvenuti, et al., Boll Soc It Biol Sper 44, 809-813 (1968); Lambert, et al., Curr Med. Chem. 9, 663-74 (2002)], which are associated with the down-regulation of pro-inflammatory proteins such as nitric oxide synthase (NOS) [Costa, et al., Br J Pharmacol 137, 413-20. (2002)]. Two models of acute inflammation were used to examine whether such effects are mediated through PPARα. First, paw edema was induced in C57B16J wild-type and PPARα^{-/-} mice by local injection of the irritant, carrageenan [Cirino, et al., Eur J Pharmacol 166, 505-10 (1989)]. Concomitant administration of either PEA (10 mg-kg⁻¹, i.p.) or WY-14643 (20 mg-kg⁻¹, i.p.) decreased edema in wild-type mice, but not in PPARα^{-/-} mutants (FIG. 2 a-b). Next, ear edema was produced in CD1 mice by topical administration of the phorbol ester, 12-O-tetradecanoylphorbol-13-acetate (TPA) [Sheu, et al., J Invest Dermatol., 94-101. (2002)]. Edema formation was attenuated by co-application of PEA (ED₅₀ 8.9 μg per ear; n=5) GW7647 or Wy-14643 (FIG. 2c). In a parallel set of experiments, the effects of PEA (20 μg) on TPA-induced edema were tested in C57B16J wild-type and PPARα^{-/-} mice. Although TPA caused similar inflammatory responses in the two strains, PEA was effective only in wild-type animals (FIG. 2 d). Interestingly, GW7647 retained a residual, albeit weak anti-inflammatory activity in PPARα^{-/-} mice (FIG. 5 b). This might be due to the ability of this compound to interact with PPARβ/δ (EC₅₀=6.2 μM) and PPARγ (EC₅₀=1.1 μM), which are also involved in the control of inflammation [Brown, et al., Bioorg Med Chem Lett 11, 1225-7 (2001)]. Together, these findings indicate that PEA reduces acute nociception and inflammation through activation of PPARα.

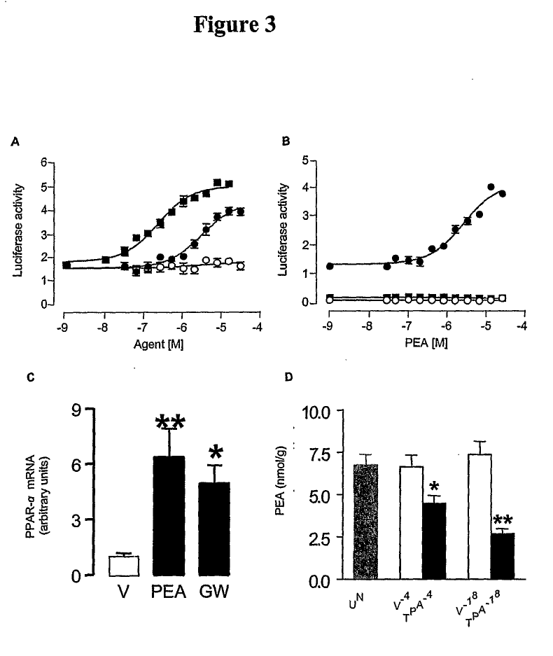

PEA activation of PPARα was investigated in vitro and in vivo. HeLa cells were engineered to stably express a luciferase reporter gene together with the ligand-binding domain of human PPARα fused to the yeast GAL4 DNA-binding domain [Willson, et al., J Med Chem 43, 527-50 (2000)]. In transactivation assays, PEA activated PPARα with a half-maximal effective concentration (EC₅₀) of 3.0 μM (n=12) (FIG. 3a). Under identical conditions, palmitic acid had no effect, while GW7647 engaged the receptor with an EC₅₀ values of 120±1 nM, respectively (FIG. 3a). The effects of PEA were isoform selective, as the compound did not activate PPARβ/δ or PPARγ at concentrations as high as 20 μM (FIG. 3b). Second, the effects of PEA on induction of PPARα mRNA expression in inflamed mouse skin was examined. Topical administration of PEA (450 μg) or GW7647 (755 μg) to the abdominal skin of C57BL6/J mice attenuated TPA-induced edema and caused a parallel increase in PPARα mRNA expression (FIG. 3 c). Consistent with these results, high-performance liquid chromatography (HPLC/MS) analyses revealed that, after topical application, PEA reached skin concentrations that were sufficient to fully activate PPARα (control (acetone): 10.4±1.3 nmol/g; PEA (450 μg): 250.2±22.4 nmol/g; n=5).

To assess whether these endogenous tissue levels of PEA are physiologically relevant, the PEA content of normal and inflamed abdominal skin was measured in C57BL/6J mice. Normal skin contained on average 6.8±0.6 nmol of PEA per g of tissue (n=4-5). Assuming an even distribution of PEA throughout the tissue volume, this content translates into a concentration that would produce maximal activation of PPARα in vitro (FIG. 3a). Importantly, TPA-induced inflammation was associated with a dramatic, time-dependent decrease in skin PEA content (FIG. 3d). A similar response was observed with OEA, another PPARα ligand, but not with anandamide, an endogenous cannabinoid agonist (data not shown).

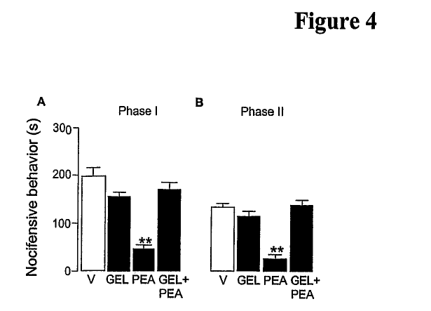

PEA and other PPARα agonists reduce nocifensive behaviors within minutes of administration—a time-course that implies a non-transcriptional mechanism of action. Though non-genomic actions of PPARα have been documented [Fu et al., Nature 425: 90-93 (2003)], their molecular bases remain elusive. Prior to ligand binding, PPARα exists in cells as a heteromeric multiprotein complex that includes the heat-shock protein, hsp90 [Sumanasekera et al., 2003 a, b]. Because Hsp90 participates in the rapid, non-transcriptional effects of estrogen hormones [Russell, et al., J Biol Chem 275, 5026-30 (2000); Bucci, et al., Br J Pharmacol 135, 1695-700 (2002)], we hypothesized that it also may be involved in PPARα mediated anti-nociception. To test this idea, we used the antibiotic geldanamycin, which selectively binds to hsp90 and disrupts its interactions with client proteins [Neckers, Drug Resist Updat 3, 203-205 (2000)]. Low doses of geldanamycin (1-10 μg, i.pl.) did not affect formalin-evoked nocifensive behavior in Swiss mice (FIG. 4a-b). However, the hsp90 inhibitor abolished the anti-nociceptive response to a maximal dose of PEA (50 μg i.pl.)(FIG. 4a-b).

Animals. Male C57BL/6J mice (25-30 g) and Swiss mice (20-25 g) were used. All procedures met the National Institutes of Health guidelines for the care and use of laboratory animals, and those of the Italian Ministry of Health. We purchased male C57BL/6J, PPARα^{-/-} (129S4/SvJae-Ppara^{tmlGonz}) and wild-type C57BL/6J mice (Jackson Laboratories or Taconic). Animals were maintained on a 12-h/12-h light/dark cycle and had free access to water and RMH 2500 chow (Prolab). For writhing tests the animals were starved overnight.

Chemicals. GW501516 was synthesized by modifications to published procedures [Chao, 2001] and fatty-acid ethanolamides as described [Giuffrida, et al., Anal Biochem 280, 87-93 (2000)]. Palmitic acid was purchased from Nu-Check Prep (Elysian, Minn.), SR141716A (rimonabant) and SR144528 were provided by RBI (Natick, Mass.) as part of the Chemical Synthesis Program of the National Institutes of Health. All other chemicals were obtained from Tocris (Avonmouth, UK) or Sigma (St. Louis, Mo.). Fresh drug solutions were prepared immediately before use in 0.9% sterile saline/polyethylene glycol/Tween 80 (90/5/5).

### Transactivation Assays

Transactivation assays were performed as previously described [Fu et al., Nature 425:90-93 (2003)].

### Formalin-Evoked Hind Paw Licking

Mice received intraplantar injections of formalin (5% vol/vol, 10 μl) in saline containing polyethylene glycol (5%) and Tween 80 (5%). The duration of paw licking was monitored by an observer for periods of 0-15 min (early or first phase) and 15-45 min (late or second phase) immediately after formalin administration. Initial tests determined that the presence of polyethylene glycol or Tween 80 did not significantly affect the formalin response.

### Writhing Tests

Mice received a single dose of magnesium sulfate (120 mg/kg, dissolved in 0.5 ml saline, i.p.) and subsequent writhing episodes were monitored for a period of 30 min thereafter. Drugs were administered in saline/Tween 80/polyethylenglycol, (90/5/5) subcutaneously, 30 minutes before magnesium sulfate.

### Irritant Contact Dermatitis

As previous described procedure [Sheu, et al., J Invest Dermatol., 94-101. (2002)], 12-O-tetradecanoylphorbol-13-acetate (TPA) was dissolved in acetone (0.03%, w v⁻¹) and topically delivered with a pipette to the inner and outer surfaces (10 μl/side) of mice ears. Experimental agents were dissolved in acetone and delivered topically (20 μl/side) 45 minutes and 4 hours after TPA. 18 hours after TPA administration, the mice were euthanized with CO₂ and the ears punched (diameter=2 mm) four times. The 4 excised punches of skin were combined and weighed to assess edema.

### Carrageenan-Evoked Paw Edema

An inflammatory response was initiated by injecting 2% (w v⁻¹, 20 μl) λ-carrageenan in sterile saline into mice hind paws using a 27-gauge needle. Drugs were administered 30 minutes before carrageenan (i.p.). Paw edema was measured using a plethysmometer (Ugo Basile, Italy).

### Statistical Analyses

Results are expressed as the mean ±s.e.m. of n experiments. The significance of differences between groups was analyzed by a one-way analysis of variance (ANOVA) followed by a Dunnett's test for multiple comparisons. Within group analysis was analyzed with a Student's t-test. Analyses were done with GraphPad Prism software (GraphPad Software, San Diego, Calif.).

### Example 2

### Use of PPARα Agonists to Decrease Neuropathic Hyperalgesia Following Partial Sciatic Nerve Injury in the Rat

This prophetic example illustrates the topical use of PPARα agonists to treat pain that is primarily not the result of inflammation, e.g. neuropathic pain. Male Sprague-Dawley rats (180-250 g) are anaesthetised with 50 mg/kg i.p. pentobarbitone sodium (Nembutal). The unilateral common sciatic nerve is exposed high in the thigh and ⅓-½ of the nerve trunk is carefully separated and tightly ligated using a siliconised silk suture (Ethicone 8-0). Then the wound is closed and the animals are allowed to survive for 8 days. During this period, signs of spontaneous pain (holding the legs in elevated position) and mechano-nociceptive hyperalgesia develop. Mechano-nociception of the hindpaws is measured by Randall-Selitto test using the Ugo Basile analgesimeter. Continuously increasing pressure is applied on the paw of conscious rats and the threshold force which elicits withdrawal is determined. The results indicate force measured on a scale calibrated in grams. Control values are measured before operation during a period of 3-4 days. Four measurements can be made on each rat and the average of the last two assessments can be taken as controls. Significant decreases in mechanical threshold should develop within 7 days after the surgery. Measurements on the 7th day are taken 1.5-2 h before and 30 min after topical administration (on the effected paw) of the PPARα agonists PEA, OEA, GW-7647 or WY-14643. The appropriate solvent(s) or vehicle(s) can also topically applied on rats to serve as a control. Changes of mechano-nociceptive thresholds in percentage compared to the respective preoperation values before and 30 min after drug administration can be calculated. Administration of the PPARα agonists should show that PPARα agonists reduce the hyperalgesia associated with neuropathic pain.

### Example 3

### Combination of CB1 and PPARα Agonists Decrease Neuropathic Hyperalgesia Following Partial Sciatic Nerve Injury in the Rat

This prophetic example further illustrates the topical administration of the combination of a PPARα agonist and a CB1 agonist to treat pain. Partial sciatic nerve injury is created and mechano-nociception of the hind paws is measured in male Sprague-Dawley rats as described in Example 2. Measurements on the 7th day are taken 1.5-2 h before and 30 min after topical administration of the combination of PPARα agonists and CB1 agonists. Combinations of a single CB1 agonist and a single PPARα agonist are administered topically (on the effected paw). CB1 agonists that are used include anandamide (AEA), WIN-55212-2 and HU-210. PPARα agonists that are used include PEA, OEA, GW-7647 and WY-14643. The appropriate solvent(s) is also topically applied on rats to serve as a control. Changes of mechano-nociceptive thresholds in percentage compared to the respective preoperation values before and 30 min after drug administration can be calculated. The administration of the PPARα agonists should reduce the hyperalgesia associated with neuropathic pain.

### Example 4

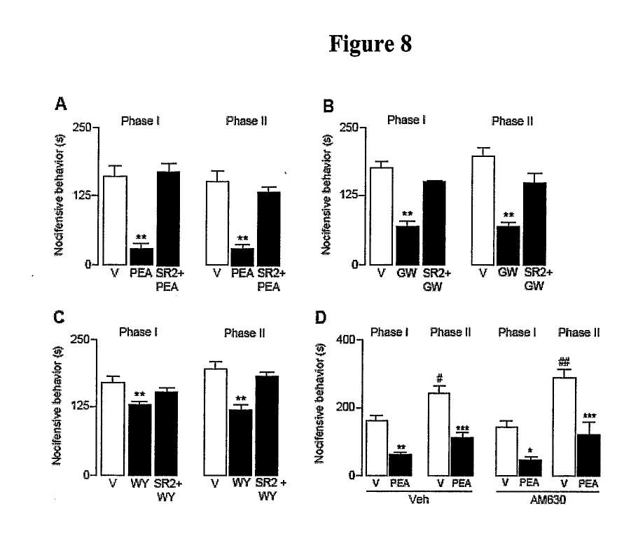

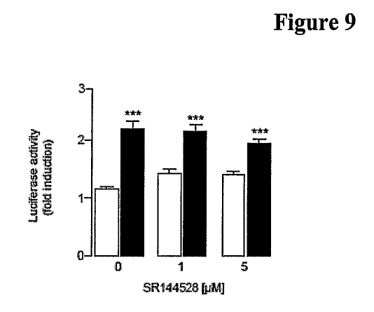

As disclosed above, the antinociceptive effect of PEA (ED₅₀=7.4 μg, i.pl.) in the formalin model (FIG. 1A,B) was absent in PPARα^{-/-} mice (FIG. 1F). Previous studies have shown that these actions are also blocked by the CB₂ cannabinoid antagonist SR144528, although PEA does not bind to CB₂ receptors (Calignano, et al., Nature 394, 277-81 (1998); Calignano, et al, Eur J Pharmacol 419, 191-8. (2001); Farquhar-Smith, et al., Pain 97, 11-21. (2002). Those reports are confirmed herein (FIG. 8 A; Table 1) and SR144528 (1 mg-kg⁻¹, s.c.) is further reported to reverse the antinociceptive effects of GW7647 and Wy-14643 (FIG. 8 B,C). However, like PEA, these PPARα agonists exhibited no significant binding to CB₂ (Table 1). Moreover, a CB₂ antagonist chemically unrelated to SR144528, AM630 (Malan et al., Pain, 93, 239 (2001), did not affect the response to PEA or GW7647 (FIG. 8 D) at a dose (2 mg-kg⁻¹, i.p.) that fully blocks CB₂ receptors in vivo (Ibrahim et al., Proc Natl Acad Sci USA, 100, 10529 (2003)), implying that SR144528 may interact with a site distinct from CB₂. This hypothetical site remains unidentified, but is likely to be located downstream of PPARα because SR144528 did not significantly antagonize PEA-induced PPARα activation in vitro (FIG. 9).

TABLE 1
Binding of PPARα agonists to human CB₂.

| | Concentration | [³H]-WIN-55212-2 Binding |
|---|---|---|
| Agent | [μM] | (% of control) |
| Vehicle | — | 100.0 ± 1.9 |
| GW7647 | 1 | 102.1 ± 4.0 |
| | 10 | 91.6 ± 4.1 |
| PEA | 1 | 106.2 ± 2.5 |
| | 10 | 98.5 ± 4.2 |
| Wy-14643 | 1 | 104.2 ± 3.6 |
| | 10 | 105.6 ± 1.5 |
| WIN-55212-2 | 1 | 4.2 ± 2.2** |

**P < 0.01, ANOVA, followed by Dunnett's post-hoc test (n = 8-9).

Next, the effect of PPARα agonists on the nocifensive behavior elicited by intraperitoneal injection of magnesium sulfate in mice, a model of visceral pain (Calignano, et al, Eur J Pharmacol 419, 191-8. (2001)), was explored. Subcutaneous administration of GW7647 (1-30 mg-kg⁻¹), Wy-14643 (4-80 mg-kg⁻¹) or PEA (1-20 mg-kg⁻¹) dose-dependently decreased the number of writhing episodes evoked by magnesium sulfate (120 mg-kg⁻¹) (FIG. 1H). Average ED₅₀ values were 7.5 mg-kg⁻¹ for GW7647, 25 mg-kg⁻¹ for Wy-14643 and 9.9 mg-kg⁻¹ for PEA (n=8) As observed in the formalin test, PEA did not significantly modify magnesium sulfate induced writhing in PPARα^{-/-} mice (FIG. 1I). Collectively, these results indicate that PPARα agonists inhibit acute nociception through selective activation of PPARα.

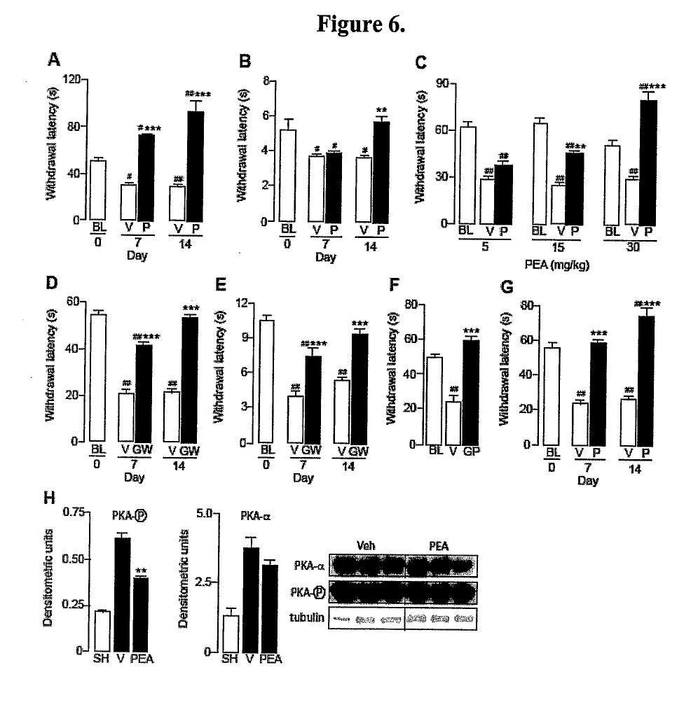

To examine whether PPARα agonists modulate the persistent pain hypersensitivity associated with nerve injury, peripheral neuropathy was produced in mice by partially ligating the left sciatic nerve, a surgical procedure that results in the development of mechanical and thermal hyperalgesia in the operated limb (Bennett et al., Pain, 33, 87 (1988)). On the 7^{th} and 14^{th} day after surgery, when pain behavior had reached maximal levels, we administered a single subcutaneous dose of PEA (30 mg-kg⁻¹) or vehicle and, 60 min later, measured mechanical (Randall et al., Arch Int Pharmacodyn Ther, 111, 409 (1957)) and thermal (Hargreaves et al., Pain, 32, 77 (1988)) pain thresholds. Administration of PEA caused a reversal of neuropathic hypersensitivity, whereas vehicle had no such effect (FIG. 6A, 6B). This antihyperalgesic response was dose-dependent (FIG. 6C), was mimicked by the synthetic PPARα agonist GW7647 (30 mg-kg⁻¹, s.c.) (FIG. 6D, 6E) and was comparable in magnitude to that produced by gabapentin (FIG. 6F), an anticonvulsant drug widely used in the clinic to treat neuropathic pain (Bennett et al., Palliat Med, 18, 5 (2004)). Like gabapentin, PEA and GW7647 did not affect mechanical and thermal pain thresholds in the non-ligated paw of neuropathic mice (data not shown).

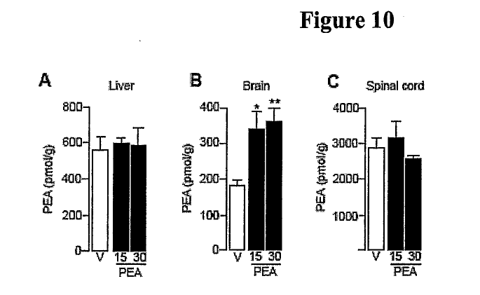

Opiate analgesics have a high liability to produce tolerance, a progressive loss of pharmacological activity caused by repeated drug administrations (Waldhoer et al., Annu Rev Biochem, 73, 953 (2004)). To assess whether tolerance develops to the antihyperalgesic effects of PPARα agonists, PEA (30 mg-kg⁻¹, s.c.) was administered once a day for 14 consecutive days from the time of surgery, measuring mechanical and thermal hyperalgesia at day 7 and 14. Mice receiving daily PEA injections exhibited significantly higher mechanical sensitivity thresholds (FIG. 6G) and brain PEA levels (FIG. 10) than did control, vehicle-treated mice. Threshold values in the group subchronically treated with PEA were similar to those of mice that had received a single PEA injection (FIG. 6A), and remained significantly higher than those of vehicle-treated mice until the 14^{th} day of the experiment (FIG. 6G). Furthermore, immunoblot analyses of lumbar spinal cord extracts revealed that phosphorylation of protein kinase A (PKA)-IIα regulatory subunit at Ser⁹⁶—which is enhanced in neuropathic mice (FIG. 6H) (Miletic et al., Neurosci Lett, 360, 149 (2004)) and may contribute to central sensitization (Kawasaki et al., J Neurosci, 24, 8310 (2004))—was reduced by PEA treatment (FIG. 6H). Spinal tissue levels of PKA α catalytic subunit were also slightly decreased by PEA, but this change did not reach statistical significance (FIG. 6H).

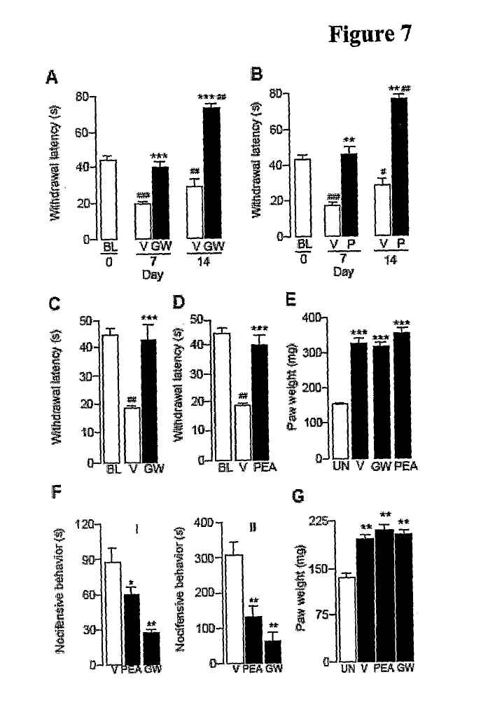

The ability of PPARα agonists to attenuate hyperalgesia was further explored in two mouse models of subchronic inflammation: (i) experimental arthritis elicited by intradermal injection of complete Freund's adjuvant (CFA) into the base of the tail (Whiteley et al., Current Protocols in Pharmacology, 2, 5.5.1-5.5.5. (2001)); and (ii) prolonged oedema induced by repeated carrageenan injections into the paw (Otterness et al., Methods Enzymol, 162, 320 (1988)). On day 7 and 14 after CFA injection, when arthritis in both hind limbs had fully developed, either GW7647 or PEA (30 mg-kg⁻¹, s.c.) was administered 60 min before measuring pain thresholds. Both drugs, but not their vehicle, suppressed mechanical (FIG. 7A, 7B) and thermal (data not shown) sensitivity. Likewise, injections of either GW7647 or PEA (30 mg-kg⁻¹, s.c.) on the third day of carrageenan treatment normalized mechanical sensitivity thresholds (FIG. 7C, D) while having no effect on paw oedema (FIG. 7E). The latter finding shows that PPARα agonists can reduce hyperalgesia independently of their well-recognized ability to alleviate inflammation (Daynes et al., Nat. Rev. Immunol. 2, 748 (2002); Taylor et al., Inflammation, 26, 121 (2002). This dissociation was confirmed in the formalin model (FIG. 7F) where PPARα-mediated antinociception was not accompanied by overt signs of anti-inflammatory activity (FIG. 7G).

These results indicate that high-affinity agonists of PPARα can selectively interact with this nuclear receptor to produce profound antinociceptive and antihyperalgesic effects. The breadth of such effects—which are herein documented using five mechanistically diverse animal models of pain—suggests that PPARα agonists may act at multiple stages of the pain-processing axis, possibly involving multiple mechanisms of action. This idea tallies well with the presence of PPARα in neurons and astrocytes of the peripheral and central nervous systems (Moreno, et al. Neuroscience, 123, 131 (2004), as well as in non-neural cells sensitive to noxious stimuli, such as keratinocytes (Khodorova et al., Nat Med, 9, 1055 (2003)). In this regard, it is noteworthy that PPARα expression in the spinal cord is enhanced during CFA-induced arthritis (Benani et al., Neurosci Lett, 369, 59 (2004)), a condition in which PPARα agonists exert striking antihyperalgesic effects (FIG. 7A,B).

These findings thus expand the therapeutic opportunities offered by high-affinity modulators of PPARα activity, a class of drugs that is now in late-stage development for the treatment of blood glucose and lipid abnormalities (Miyachi, IDrugs, 7, 745 (2004)). For example, according to the present invention, dual PPAR modulators combining the analgesic properties of PPARα agonists with the insulin-sensitizing properties of PPARγ agonists could offer an innovative therapeutic approach to painful diabetic neuropathies, a condition that afflicts 6 million people in the US alone.

Chemicals. GW7647 was a kind gift of Professor G. Tarzia and Dr. B. Di Giacomo (University of Urbino, Italy). PEA was prepared as described (Giuffrida, et al., Anal Biochem 280, 87-93 (2000)). SR144528 was provided by RBI (Natick, Mass.) as part of the Chemical Synthesis Program of the National Institutes of Health (NIH). All other chemicals were from Tocris (Avonmouth, United Kingdom) or Sigma (St. Louis, Mo.). Fresh drug solutions were prepared immediately before use in 0.9% sterile saline or in a vehicle of 0.9% sterile saline/5% polyethylene glycol/5% Tween 80.

Animals. We used male C57BL6 (25-30 g) or Swiss mice (20-25 g). All procedures met the NIH guidelines for the care and use of laboratory animals, and those of the Italian Ministry of Health (D.L. 116/92). We purchased male C57BL6, PPAR-α^{-/-} (129S4/SvJae-Ppara^{tmlGonz}) and wild-type C57BL6 mice from Taconic (Germantown, N.Y.) or Jackson Labs (Bar Harbor, Me.). The mice were maintained on a 12-h/12-h light/dark cycle with free access to water and chow (RMH 2500, Prolab, Framingham, Mass.).

### Pain Assays

Formalin test. Formalin (5%, 10 μl)-induced paw licking was monitored for 0-15 min (first phase) and 15-45 min (second phase) as described (Calignano, et al., Nature 394, 277-81 (1998).

Writhing test. Magnesium sulfate was injected (120 mg-kg⁻¹ in 0.5 ml saline, i.p.) in 12-h food-deprived mice and measured writhing episodes for 30 min (Calignano, et al, Eur J Pharmacol 419, 191-8. (2001)). Drugs or vehicle were administered by subcutaneous injection 30 min before magnesium sulfate.

Carrageenan-induced paw oedema. An inflammatory response was initiated in the left hindpaw of male Swiss mice by injecting %-carrageenan (1% weight-vol⁻¹ in sterile saline, 50 μl) using a 27-gauge needle, and maintained it by repeating the injections on day 3, 7 and 14 after the first administration. Paw oedema was measured using a mouse plethysmometer (Ugo Basile, Italy) or by weighing the excised paw.

Sciatic nerve ligation was performed essentially following the method of Bennett and Xie (Bennett et al., Pain 33, 87 (1988)). Briefly, male Swiss mice were anesthetized with sodium pentobarbital, the left sciatic nerve was exposed at mid-thigh level through a small incision and one-third to one-half of the nerve thickness was tightly ligated with a silk thread. The wound was closed with a single muscle suture and skin clips, and dusted with streptomycin powder. In sham-operated animals, the nerve was exposed but not ligated.

Adjuvant-induced arthritis. 0.1 ml of complete Freund's adjuvant (Mycobacterium tuberculosis, Sigma) was intradermally injected into the base of the tail of male Swiss mice. This procedure resulted in significant inflammation of both hind paws, which was monitored visually or with a mouse plethysmometer. The hyperalgesia measurements reported here are those of the left hind paw, but similar readings were obtained in the contralateral limb.

Mechanical hyperalgesia was determined by measuring paw withdrawal thresholds to an increasing pressure onto the dorsal surface of the paw using an analgesimeter (Ugo Basile, Italy) with a cut-off of 250 g. Withdrawal thresholds were measured on both the ipsilateral paw (inflamed or ligated) and contralateral paw (uninflamed or unligated) 60 min after subcutaneous drug administration.

Thermal hyperalgesia was assessed by the methods of Hargreaves (Hargreaves et al., Pain, 32, 77 (1988)) by measuring the latency to withdraw the hind paws from a focussed beam of radiant heat applied to the plantar surface, using an appropriate apparatus (Ugo Basile, Italy). The cut-off time was set at 30 sec. The mice were habituated to the apparatus for 10-15 min two days prior to the experiment.

Receptor binding. CB₂ receptor binding assays were conducted in membranes of CB₂-overexpressing CHO Cells (Perkin-Elmer, Boston, Mass.) using [³H]Win-55212-2 (Perkin-Elmer, 40-60 Ci/mmol, 10 nM) as a ligand (Tarzia et al., J Med. Chem. 46(12):2352 (2003)).

PPAR-α transactivation. Plasmids were generated which contained the ligand-binding domain of human PPAR-α (nucleotides 499-1,407) fused to the DNA-binding domain of yeast GAL4 under control of the human cytomegalovirus promoter and to a neomycin resistance gene to provide stable selection with 0.2 mg-ml-1 G418 (Calbiochem). We cultured HeLa cells in Dulbecco's-modified Eagles's medium (DMEM) supplemented with fetal bovine serum (10%), transfected them with Fugene 6 (3 ml, Roche) containing the pFR-luc plasmid (1 mg, Stratagene) and, 18 h later, replaced the media with DMEM containing hygromycin (0.1 mg ml₋₁, Calbiochem). After 4 weeks, we isolated surviving clones, analysed them by luciferase assay, and selected for transfection a cell line that demonstrated highest levels of luciferase activity. Cells were maintained in DMEM containing hygromycin and G418. For transactivation assays, we seeded cells in 6-well plates and incubated them for 7 h in DMEM containing hygromycin and G418, plus appropriate concentrations of test compounds. We used a dual-luciferase reporter assay system (Promega) and an MLX Microtiter® plate luminometer (Dynex) to determine luciferase activity in cell lysates.

Immunoblot analysis. Lumbar spinal cord tissue was weighed, homogenized in Tris-HCl (20 mM, pH 7.5), 10 mM NaF, 150 mM NaCl, 1% Nonidet P-40, 1 mM PMSF, 1 mM Na₃VO₄, leupeptin and trypsin inhibitor (10 μg/ml). After 1 h, the tissue lysates were subjected to centrifugation ay 100,000×g for 15 min at 4° C. Protein concentrations were estimated using a Bio-Rad protein assay and bovine serum albumin as a standard. Equal amounts of protein (20 μg) were dissolved in Laemmli sample buffer, boiled for 5 min, subjected to sodium dodecyl sulphate-polyacrylamide gel electrophoresis (8% polyacrylamide) and transferred onto nitrocellulose membranes at 240 mA for 40 min at room temperature. The filter was blocked with PBS, 5% non-fat dried milk for 40 min at room temperature and incubated overnight at 4° C. with rabbit primary antibodies to phosphorylated Ser⁹⁶ PKA regulatory subunit (Upstate Biotechnology, New York), PKAα catalytic subunit (Santa Cruz Biotechnology, California), or α-tubulin (Sigma, Mo.) at a dilution of 1:500 for 1 h at room temperature in PBS, 5% non-fat dried milk, 0.1% Tween 20. After washing and incubation with peroxidase conjugate secondary antibody (anti-rabbit IgG-horseradish, 1:2,000 dilution) for 1 h at room temperature, the blots were developed using enhanced chemiluminescence detection reagents (Amersham) according to the manufacturer's instructions, and exposed to Kodak X-Omat films. The films were scanned and densitometrically analysed with a model GS-700 imaging densitometer.

LC/MS analysis. PEA was measured by high-performance liquid chromatography/tandem mass spectrometry. Tissue samples were weighed, cut into 1 mm² pieces, and incubated with 1 mL of acetonitrile at 4° C. overnight. Samples of the incubates (100 μl) were transferred to 96-well plates, diluted with 250 μL of acetonitrile containing [H₂]-ethanolamine-OEA as an internal standard, and injected into the HPLC. HPLC analyses were carried out on a Waters 2790 Alliance system (Milford, Mass.) using a Phenomenex Synergi Polar-RP column (2 mm×150 mm, 4μ; Torrance, Calif.) and a mobile phase of 0.1% formic acid in water (solvent “A”) and 0.1% formic acid in acetonitrile (solvent “B”). Run conditions were isocratic (25% A and 75% B), at a flow rate of 0.3 mL min⁻¹, column temperature of 45° C., and a run time of 3 minutes. The HPLC system was interfaced with a Micromass Ultima (Beverly, Mass.) tandem mass spectrometer. The samples were analyzed using an electrospray probe in the positive ionization mode with the cone voltage set at 40 V and capillary at 3.2 kV. The source and desolvation temperature settings were 130° C. and 500° C., respectively. The voltage of the CID chamber was set at −17 eV. Multiple reaction monitoring was used for the detection of PEA as [M+H]⁺ (m/z 300>62) and the standard as [M+H]⁺ (m/z 330>66).

### Statistical Analyses

Results are expressed as the mean ±s.e.m. of n experiments. All analyses were conducted using the GraphPad Prism software (GraphPad Software, San Diego, Calif.). The significance of differences between groups was determined by one- or two-way analysis of variance (ANOVA) followed by a Dunnett's or Bonferroni's test for multiple comparisons, as appropriate. Within group analysis was conducted with a Student's t-test.

This application contains subject matter related to that of U.S. patent application Ser. No. 10/112,509 filed Mar. 27, 2002; U.S. Provisional Application No. 60/336,289 filed Oct. 31, 2001; U.S. Patent Application No. 60/279,542 filed Mar. 27, 2001, U.S. Provisional Application No. 60/485,062 filed Jul. 2, 2003, and U.S. patent application Ser. No. 10/681,858 filed Oct. 7, 2003. The contents of each of the above-referenced applications is incorporated herein by reference in its entirety.

Each of the patents, published patent applications, and other publications cited in this specification are incorporated by reference in their entireties to the extent not inconsistent with the present disclosure and in addition with particular reference to the compounds and pharmaceutical compositions they disclose.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating a mammalian subject having a non-inflammatory pain or a pain initiated or caused by a primary lesion or dysfunction of the nervous system, said method comprising administering to the subject a PPARα agonist in a therapeutically effective amount with the proviso that the PPARα agonist is other than palmitylethanolamide (PEA).

2. The method of claim 1, wherein the pain is selected from the group consisting of post trigeminal neuralgia, neuropathic low back pain, peripheral or polyneuropathic pain, complex regional pain syndrome, causalgia, and reflex sympathetic dystrophy, diabetic neuropathy, toxic neuropathy, cancer, and neuropathy caused by chemotherapeutic agents.

3. The method of claim 1, wherein the pain is a neuropathic form of allodynia or hyperalgesia.

4. The method of claim 1, wherein the primary lesion or dysfunction of the nervous system is caused by a mechanical injury to a nerve of the subject.

5-13. (canceled)

14. The method of claim 1, wherein the primary lesion or dysfunction of the nervous system is diabetic neuropathy.

15. The method of claim 1, wherein the subject is human.

16. (canceled)

17. The method of claim 1, wherein the PPARα agonist is a fatty acid amide which is not a cannabinoid receptor agonist.

18. The method of claim 1, wherein the PPARα agonist is OEA.

19-31. (canceled)

32. The method of claim 1, wherein the method further comprises administration of a CB₁ cannabinoid receptor agonist.

33-34. (canceled)

35. The method of claim 1, wherein the PPARα agonist is OEA.

36. (canceled)

37. The method of claim 1, wherein a FAAH inhibitor is also administered.

38-42. (canceled)

43. The method of claim 1, wherein the PPARα agonist is not a cannabinoid receptor agonist.

44. (canceled)

45. The method of claim 1, wherein the PPARα agonist is not an activator, agonist, or antagonist of the vanilloid receptor.

46. The method of claim 1, wherein the PPARα agonists is a high affinity PPAR α agonist.

47. The method of claim 46, wherein the PPARα agonist is a fatty acid alkanolamide.

48. The method of claim 46, wherein the PPARα agonist is other than a fatty acid alkanolamide.

49-51. (canceled)

52. The method of claim 1, wherein an anandamide transport inhibitor or FAAH inhibitor is also administered to the subject.

53. The method of claim 1, wherein the PPARα agonist is not an activator, agonist, or antagonist of the vanilloid receptor.

54-58. (canceled)

59. A pharmaceutical composition comprising:
a first agent which is a PPARα agonist other than PEA,
a second agent which is a CB1 cannabinoid receptor agonist, a FAAH inhibitor, or an anandamide transport inhibitor; and
a pharmaceutically acceptable excipient.

60. The composition of claim 59, wherein the CB1 agonist is anandamide.

61. The composition of claim 59, wherein the agonist is OEA.

62. The composition of claim 59, wherein the CB1 receptor agonist is selective for the CB1 receptor over the CB2 cannabinoid receptor.

63-64. (canceled)

65. The pharmaceutical composition of claim 59, wherein the PPARα agonist is not a substrate for FAAH.

66. (canceled)
